(19) United States
(12) Patent Application Publication
(10) Pub. No.: US 2022/0367064 A1
(43) Pub. Date: Nov. 17, 2022

(54) Systems and Methods for Fertility Prediction and Increasing Culling Accuracy and Breeding Decisions

(71) Applicants: Membrane Protective Technologies, Inc., Fort Collins, CO (US); Acuity Ag Solutions, LLC, Carlyle, IL (US)

(72) Inventors: Clint Schwab, Mascoutah, IL (US); Lisa A. Herickhoff, Fort Collins, CO (US); Amanda Minton, Bedford, IN (US); Christopher Bennett, Dallas, TX (US); Caleb Schull, Pinckneyville, IL (US)

(21) Appl. No.: 17/765,741

(22) PCT Filed: Sep. 30, 2020
(86) PCT No.: PCT/US20/53651

§ 371 Date: Mar. 31, 2022

Related U.S. Application Data
(60) Provisional application No. 62/908,743, filed on Oct. 1, 2019.
(60) Provisional application No. 63/049,608, filed on Jul. 8, 2020.

(51) Int. Cl. G16H 50/30; G01N 33/68; G01N 15/14; G16H 50/70

(52) U.S. Cl. CPC G16H 50/30; G01N 33/689; G01N 15/147; G16H 50/70; G01N 2015/1006

(57) ABSTRACT

Embodiments of the present invention provide predictions from semen qualities (7) embryo characteristics (9), qualities (11), intracellular qualities (13), extracellular qualities (14), or the like of which a computational device prediction models automated computational transformation algorithm (3) may be applied to create a prediction model transformed data (4) perhaps to generate a prediction models completed prediction output which may be used to predict parameters (6) such as fertility-related parameters, fertility of an animal, embryo success rate, or the like.

### PRIORITY CLAIM

This is a PCT International Patent Application claiming priority to and the benefit of U.S. Provisional Application Nos. 63/049,608 filed Jul. 8, 2020 and 62/908,743 filed Oct. 1, 2019, each hereby incorporated by reference herein.

### TECHNICAL FIELD

The present invention generally relates to systems and methods for fertility prediction, utilizing characteristics of cells such as gametes, embryos, or the like for generating informed data utilized to make breeding decisions, culling decisions, or perhaps even decisions regarding the type of utilized assisted reproductive technology.

### BACKGROUND

In production agriculture it may be necessary to make decisions about which males and females to use to create future generations. These decisions may be based on the male's ability to efficiently generate offspring, to provide genetic improvement, to provide desirable characteristics to a herd, or even to provide variation. The sire's genes may be transferred via methods perhaps such as ‘live-cover’ (allowing natural copulation) or artificial reproductive techniques such as perhaps artificial insemination, in vitro fertilization, embryo transfer, intracytoplasmic sperm injection, sperm sorting or sex selection, or the like. The ultimate success of such techniques may depend perhaps on the quality of the genetics, the quality of the genetic material imparted, the external characteristics of the sperm, the technician performing the technique and other such similar characteristics in addition to the female-influenced factors, or the like.

In agriculturally important mammals, if male animals do not meet certain genetic criteria, certain phenotypic criteria or do not have adequate fertility (e.g., provide quality sperm cells) then perhaps they may be removed from the herd (culled), enter the food animal market, castrated or perhaps utilized for other such appropriate tasks or purposes including, as a “mount” in a reproductive setting, as a show animal, or the like.

Many agriculturally important animals are bred using artificial insemination. This may require sperm to be collected from the male, diluted, processed for cooling (to perhaps about 17° or even about 4° C.), or perhaps even frozen in liquid nitrogen, or the like. During this processing, and/or storage, damage may occur to the sperm cell that can change its effectiveness in fertilization. In some instances, analysis of sperm cells before processing, after thawing or warming may commonly be used to determine if the sperm cells are adequately healthy, if the storage method was effective or perhaps to decide if the cells will be viable for use in assisted reproductive technologies. However, in the past, single assay of sperm cells (either before or after freezing) have not been correlated, perhaps even highly correlated, with fertility or even success in assisted reproductive technologies.

The success or failure of breeding animals has economically important consequences for the producers and users of such animals. Consider in a herd of beef cows, for every 21 days (an average cycle) that a cow has not conceived a calf, it costs up to about $100/cow, when considering the costs associated such as feed, transportation, labor, veterinary intervention, or the like. If a producer has 1000 cows to be artificially inseminated and about 30% or more do not conceive a calf in the first insemination, the producer's costs may have increased by more than $30,000. The producer may have extra expenses during calving because he may require extra labor, extra semen, or the like since all the cows will now not calve at the same time or perhaps because the selected genetics may no longer be available. In addition, he might be required to use a herd bull instead which may increase the danger involved in sorting the cow herd and perhaps even decrease the expected genetic gain.

Because of the current prevalence of genomic testing, breeding decisions may be made solely on genetic potential (e.g., the analysis of genetic material carried by the male and the potential to pass these onto an offspring). In the past, in bovine especially, a large number of “test” breedings would be performed to evaluate the male's genetics, fertility, fecundity, and if the sire's potential could be numerically evaluated via offspring. This was viewed as a tremendously expensive endeavor and was only performed on a handful of animals predicted to have the highest potential. However, a favorable outcome was the evaluation of the male's ability to produce fertile sperm cells and a known genetic output. While genetic testing may be rapid and may allow testing of many young animals, it may not accurately predict the fertility of these animals. Today, in young bulls for example, some traits such as a propensity for weight gain can be estimated using DNA analysis or genomic testing. Unfortunately, not all traits are highly heritable and other traits can be highly affected by environmental conditions. Fertility may be a lowly heritable trait and even with increased accuracy from genomic enhanced breeding values, the accuracy can still be low. Reproductive traits tend to be especially unreliable for genetic testing and therefore one may not be able to use this to estimate the efficacy of the animal during use in live cover and in assisted reproductive technologies. Therefore, prediction based on genomic testing may be less than reliable to predict success.

In 2009, a study by Holt may suggest that perhaps a series of laboratory tests could be useful to predict fertility. However, as the author mentions, predicting if a sperm will reach an oocyte is one matter, but predicting complete fertilization, full-term pregnancy or even size of a litter requires a level of complexity that is desired but difficult. The ability to classify sperm as substandard based on a trait or even a set of physiological characteristics may be possible and may even be correlated with a characteristic of fertility such as conception rates, however they may not necessarily be predictive and may be insufficient to discriminate between animals with differing degrees of fertility. The author also notes that, “attempting to predict the odds of spermatozoa meeting an egg will therefore remain difficult as we know very little about sperm function.” And while it may be intuitive that cells having misshapen heads or tails or other physical defects may prevent it from traversing the female reproductive tract and thus rendering these particular cells ineffective, it may not be a simple answer of a single trait but perhaps a combination of traits and attributes that may enable a cell to survive and perhaps even flourish in a reproductive environment. Moreover, Amann et al. 2018 may suggest that a variety of female and male microenvironments and management factors may impact fertility but these are “difficult to measure or estimate accurately.” In fact, the authors may suggest that seeking estimates of pregnancy rates using male-to-male differences might be better “abandoned unless biological difference is large.”

It may be hypothesized that to help understand fertility potential, a physical exam may be conducted. The physical examination may be utilized to predict if the male animal is physically capable of production of sperm cells and if it appears reproductively healthy.

Beyond a physical exam, past techniques to predict fertility may include the techniques as discussed in U.S. Pat. No. 9,458,506 to Chavez, et al., wherein embryo quality, perhaps specifically embryos that are in the blastocyst stage, may be screened for the presence of gene expression levels. This technology may be primarily related to detection of aneuploidy and other such detrimental embryo defects. It may not focus on male or female gametes or decisions regarding or involving selection or differentiation of such gametes.

In U.S. Pat. No. 10,162,800 to Elashoff, et al., a system may be used for determining probability of achieving pregnancy by perhaps correlating fertility-associated phenotypic traits with pregnancy outcomes, then providing pregnancy outcome data for naïve or virgin females. These may both be substantially different from using male gamete data or even a combination of male gamete, female gamete, and/or embryo data to generate decision-making data for the specific animal being tested, for culling decisions, to determine type of artificial reproductive technologies, or the like as detailed in the embodiments of the present invention.

It may be important in agricultural production systems that animals perform to their optimum and that suboptimal animals may be quickly identified and removed from the herd. The expense of keeping suboptimal animals may negatively affect the production system success. Reproductive potential may directly impact optimum performance of an animal. Moreover, the time used in a successful breeding (e.g., parturition of a live offspring) is of utmost fiscal importance. Further, in non-agricultural animals such as perhaps horses, dogs, exotic species, humans, or the like, it may be important to understand their fertility potential so that undue expenses are not expended to test assisted reproductive technologies that would not work. Regardless of application (agricultural or non-agricultural) understanding and predicting fertility potential may allow one to target specific artificial technologies that may be more effective given the anticipated limitations in fertility. These informed decisions are fiscally responsible on many levels of all the reproductive industries.

With the benefits of known fertility, and negative impacts of infertility, delayed fertility, or decreased fecundability, it may be important to develop a technique that could enable prediction of the fertility of a dose of semen or even of an animal's overall fertility. In turn, this may enable control over purchasing, decisions regarding use of said animal for breeding within a herd, use of specific technologies that could overcome such limitations, alternative role of said animal within a herd or group, removal from a herd, or perhaps even different breeding schemes that may enable leveraging specific tools to overcome limitations.

### DISCLOSURE OF INVENTION(S)

With the inability to predict fertility and fertility-related values, which can have broad and far reaching impacts across species, it may be necessary to create methods and systems to predict fertility and even enable informed decisions on the use of a specific dose of semen, perhaps the specific male animal, or even specific artificial reproductive techniques, or even the specific male and female combination, or the like. These ultimately may lead to decisions on the fate of the male, the fate of the female, the gametes, or the like.

The present invention includes a variety of aspects, which may be selected in different combinations based upon the particular application or needs to be addressed. In various embodiments, the inventions may include systems and methods to utilize, in aggregation, combination, or even as set(s), a series of data generated from cells such as assays on male or female gametes or embryos or the like. A system may utilize data to predict fertility-related attributes or even fertility-related values, followed by data interpretation to provide actionable information. A system may enable informed decision making for reproductive and breeding decisions.

It is an object of embodiments of the present invention to provide a system and method to optimally utilize limited gamete resources, regardless of species.

It is another object of embodiments of the present invention to provide a system and method to improve the accuracy of decision making based on current analytic information rather than presumptive, or perhaps even historical data.

It is yet another object of embodiments of the present invention to provide a system and method to improve the decision-making abilities to create the optimum composition of a group of animals based on desired outputs while including inputs from disparate sources.

Naturally, further objects, goals and embodiments of the inventions are disclosed throughout other areas of the specification, claims, and drawings.

### BRIEF DESCRIPTION OF THE DRAWINGS

FIG. 1 demonstrates the predictive ability of using Combination 1 of data of boar semen quality attributes, characteristics, inputs, and physical characteristics to predict number born alive (“NBA”) in sows. A perfect correlation-line is drawn along with the predictive ability of the specific combination (r² value), or a method of prediction. FIG. 2 demonstrates the predictive ability of using Combination 1 of data of boar semen quality attributes, characteristics, inputs and physical characteristics to predict total born (“TB”) in sows. A perfect correlation-line is drawn along with the predictive ability of the specific combination (r² value), for a method of prediction

FIG. 3 demonstrates the predictive ability of using Combination 1 of data of boar semen quality attributes, characteristics, inputs and physical characteristics to predict farrowing rates in sows. The perfect correlation-line is drawn along with the predictive ability of the specific combination (r² value), for a method of prediction.

FIG. 4 demonstrates the predictive ability of using Combination 1 of data of boar semen quality attributes, characteristics, inputs and physical characteristics to predict conception rates in sows. The perfect correlation-line is drawn along with the predictive ability of the specific combination (r² value) for a method of prediction.

FIG. 5 demonstrates the effect of using Combination 2 of data of boar semen quality attributes, inputs, characteristics, and physical characteristics to predict number born alive in sows. The perfect correlation-line is drawn along with the predictive ability of the specific combination (r² value) for a method of prediction. FIG. 6 demonstrates the effect of using Combination 2 of data of boar semen quality attributes, inputs, characteristics, and physical characteristics to predict total born in sows. The perfect correlation-line is drawn along with the predictive ability of the specific combination (r² value) for a method of prediction.

FIG. 7 demonstrates the effect of using Combination 2 of data of boar semen quality attributes, inputs, characteristics, and physical characteristics to predict farrowing rates in sows. The perfect correlation-line is drawn along with the predictive ability of the specific combination (r² value) for a method of prediction.

FIG. 8 demonstrates the effect of using Combination 2 of data of boar semen quality attributes, inputs, characteristics, and physical characteristics to predict conception rates in sows. The perfect correlation-line is drawn along with the predictive ability of the specific combination (r² value) for a method of prediction.

FIG. 9 demonstrates the effect of using Combination 3 of data of boar semen quality attributes, characteristics, inputs, and physical characteristics to predict number born alive in sows. The perfect correlation-line is drawn along with the predictive ability of the specific combination (r² value) for a method of prediction.

FIG. 10 demonstrates the effect of using Combination 3 of data of boar semen quality attributes, characteristics, inputs, and physical characteristics to predict total born in sows. The perfect correlation-line is drawn along with the predictive ability of the specific combination (r² value) for a method of prediction.

FIG. 11 demonstrates the effect of using Combination 3 of data of boar semen quality attributes, characteristics, inputs, and physical characteristics to predict farrowing rates in sows.

The perfect correlation-line is drawn along with the predictive ability of the specific combination (r² value) for a method of prediction.

FIG. 12 demonstrates the effect of using Combination 3 of data of boar semen quality attributes, characteristics, inputs, and physical characteristics to predict conception rates in sows. The perfect correlation-line is drawn along with the predictive ability of the specific combination (r² value) for a method of prediction.

FIG. 13 demonstrates the effect of using quality, attributes, characteristics, inputs, and physical characteristics to predict a farrowing rate. The perfect correlation-line is drawn along with the predictive ability of the specific combination (r² value) for a method of prediction.

FIG. 14 demonstrates the effect of using quality, attributes, characteristics, inputs, and physical characteristics to predict a total born. The perfect correlation-line is drawn along with the predictive ability of the specific combination (r² value) for a method of prediction.

FIG. 15 demonstrates the effect of using quality, attributes, characteristics, inputs, and physical characteristics to predict number born alive. The perfect correlation-line is drawn along with the predictive ability of the specific combination (r² value) for a method of prediction.

FIG. 16 demonstrates the effect of using quality, attributes, characteristics, inputs, and physical characteristics to predict conception rates. The perfect correlation-line is drawn along with the predictive ability of the specific combination (r² value) for a method of prediction.

FIG. 17 shows the results of a flow cytometric assay of zinc concentration within a population of cells.

FIG. 18 shows the different populations of sperm cells with different levels of aggresomes (ubiquitin containing proteins).

FIG. 19 is a flow cytometry dot plot showing the fluorescence of individual sperm cells (individual dots=1 measured cell) when assaying for both intact acrosomes (Y-Axis) and membrane quality (X-Axis).

FIG. 20 is a flow cytometry dot plot demonstrating the identification of DNA fragmentation.

FIG. 21 is a non-limited example of a picture of a normal cell.

FIG. 22 is a non-limiting example of a picture of a cell abnormality with a distal midpiece reflex.

FIG. 23 is a non-limiting example of a picture of a cell abnormality with a coiled tail and droplet.

FIG. 24 is a non-limiting example of a picture of a cell abnormality with a proximal droplet.

FIG. 25 shows a non-limiting example of a schematic drawing in accordance with some embodiments of the present invention.

### MODE(S) FOR CARRYING OUT THE INVENTIONS

It should be understood that the present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the present invention may include a variety of parameters that can be utilized to predict various values that can enable informed decisions, predictions, methodology, fertility, and even choices in a production situation. Embodiments of the present invention may provide methods and systems for efficient fertility prediction, fertility decisions or even animal use decisions based on characteristics or qualities from semen, female information, embryo, reproductive cells, or the like.

As may be understood from FIG. 25, fertility prediction may be based on: semen qualities (7) from an ejaculate of a male animal (8); embryo characteristics (9) from an embryo (10); qualities (11) from a male animal (8) and a female animal (12); intracellular qualities (13) or even extracellular qualities (14) of a reproductive cell (15); or the like. Such information (e.g., qualities, characteristics, etc.) may be evaluated and may even be used to predict parameters (6) such as fertility-related parameters (21), fertility of an animal (22) (male or female or both), embryo success rate (23), or the like based on the information. Evaluation may include determining or even setting a value or an amount of information. Prediction of fertility-related parameters may include the foretelling of how an animal, gamete, embryo, reproductive cell or the like may affect the ability to produce offspring, reproduction, fecundity, parturition, birthrate, or the like. Such prediction may be accomplished by computational device prediction models automated computation transformation algorithms or the like.

Fertility-related parameters and other parameters may help in predicting and may help in making decisions such as but not limited to a breeding decision, a culling decision, a type of assisted reproductive technology used, doing nothing, or the like. Assisted reproductive technology may include artificial insemination, post cervical artificial insemination, deep uterine artificial insemination, in vitro fertilization, embryo transfer, intracytoplasmic sperm injection, sperm sorting or sex selection, or the like. Fertility-related parameters (21) include, but are not limited to: conception rate, parturition rate, total number of animals born alive, total number of animals born, or the like. A parturition rate may encompass the number of animals that may actually give birth, the number of animals that are identified as pregnant, and the delivery of a baby and placenta, or the like. Fertility-related parameters may include, but are not limited to, calving rate, foaling rate, farrowing rate, pregnancy rate, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, sperm quality, oocyte quality, oocyte fertility potential, oocyte health, post-thaw oocyte health, other fertility predictive measurements, any combination thereof, or the like. Fertility related parameters may include birth of mummified embryos, number of mummified embryos, or the like.

Each of the aforementioned parameters may play a role in decisions that may impact profitability of a livestock operator, a breeding operation, an assisted reproduction business, or perhaps even a betrothal situation, or the like.

Prediction analytics may utilize data, reporting or analysis, monitoring, and predictive analysis which may include gamete derived data, input data from past breedings, and/or current information, and even predicting fertility and offspring data for current breedings and the like. An action plan may be created for breeding, technique, fate of a male, fate of gametes, embryo or even females or the like.

Embodiments of the present invention may include a variety of data, attributes, values, responses or characteristics, interpretation, derived values, or the like which may be used to create predictions. Such may include, but is not limited to, gamete, gamete quality, embryo quality, animal traits, environmental information, location information, or the like. These may largely be grouped into animal information, morphology, motility, functional parameters, analysis of healthy or even unhealthy cells, antioxidant related values, sperm developmental conditions, environmental conditions, mating information, or the like. Within each of these values there may be a particular combination of categories or even sub-categories that can be utilized to provide a greater or less prediction.

The various information (2) such as qualities, characteristics, parameters, or the like may be considered a computational device data input which can be evaluated in a prediction models automated computational transformation algorithm (3) where data may be used and applied to create a prediction model transformed data (4) which may generate prediction models completed prediction output (5). Such a system can be configured by programming, firmware, algorithms, or the like. A prediction models automated computational transformation algorithm (3) may be a model such as but not limited to statistical models, mathematical models, machine learning models, regression analyses, any combination thereof, or the like. A prediction models automated computational transformation algorithm (3) may be a trained, automatically self-improving algorithm perhaps based on existing data. An algorithm can embody a set of statistical assumptions concerning the generation of sample data. A model can be specified as a mathematical relationship between one or more random variables and other non-random variables. An output (5) may provide generation of a numeric indication of a fertility-related parameter. Variances (20) may be used to predict fertility-related parameters which may include but are not limited to, variances relative to another population, variances relative to populations within a sample, variances relative to other samples taken from a same animal, variances relative to samples taken from related animals, variances within a population, any combination thereof, or the like. Generation of a numeric indication of fertility-related parameters may include any mathematical property such as but not limited to Rho correlation, delta of means, accuracy, correlation coefficients such as perhaps R², precision, contingency table, any combination thereof, or the like.

Regression analysis may provide analysis of certain values which may be used perhaps as a form of predictive modelling techniques or success that can investigate the relationship between a dependent (target) and independent variable(s) (predictor). Such analysis can be used in forecasting and even finding a causal effect relationship between the variables. A coefficient of determination (r²) may be an output of regression analysis. R² may provide a numeric indication of a fertility-related parameter. R² may be interpreted as the proportion of the variance in the dependent variable that can be predictable from the independent variable. A coefficient of determination may be the square of the correlation (r) between predicted y scores and actual y scores; thus, it can range from 0 to 1. With linear regression, the coefficient of determination may also equal to the square of the correlation between x and y scores. An r² of 0 may means that the dependent variable cannot be predicted from the independent variable. An r² of 1 means the dependent variable can be predicted without error from the independent variable. An r² between 0 and 1 may indicates the extent to which the dependent variable can be predictable. For example, an r² of 0.10 may mean that 10 percent of the variance in Y may be predictable from X; an r² of 0.20 means that 20 percent may be predictable; and so on. The formula for computing the coefficient of determination for a linear regression model with one independent variable is given below.

R²={(1/N)*Σ[(xᵢ−x)*(yᵢ−y)](σₓ*σ_{y})}²

where N is the number of observations used to fit the model, Σ is the summation symbol, xᵢ is the x value for observation i, x is the mean x value, y, is the yᵢ value for observation i, y is the mean y value, σₓ is the standard deviation of x, and σ_{y} is the standard deviation of y.

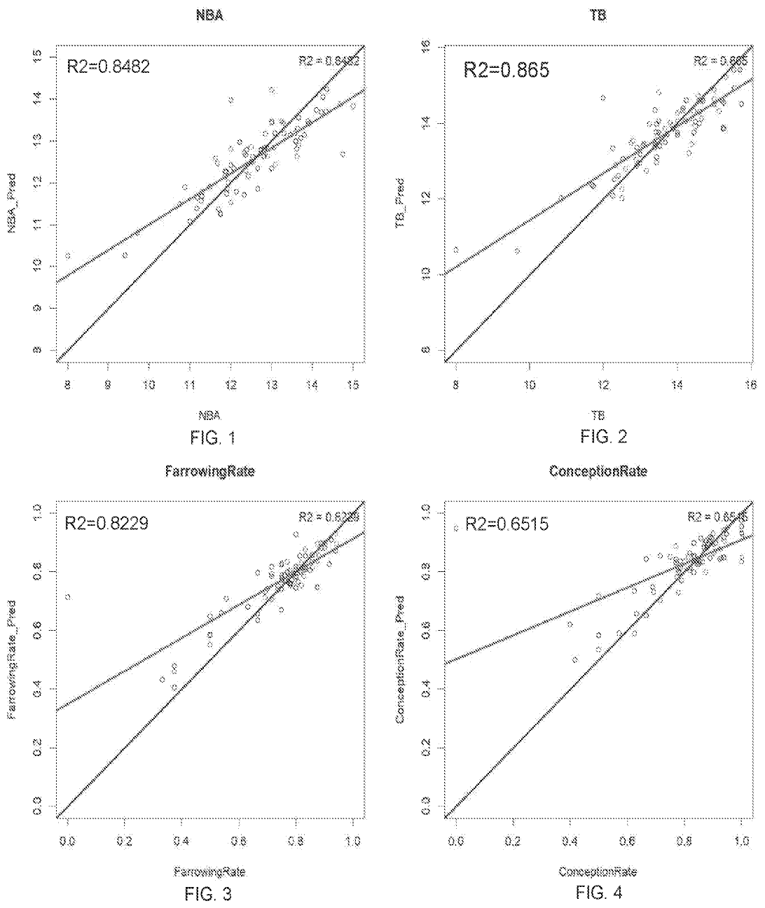

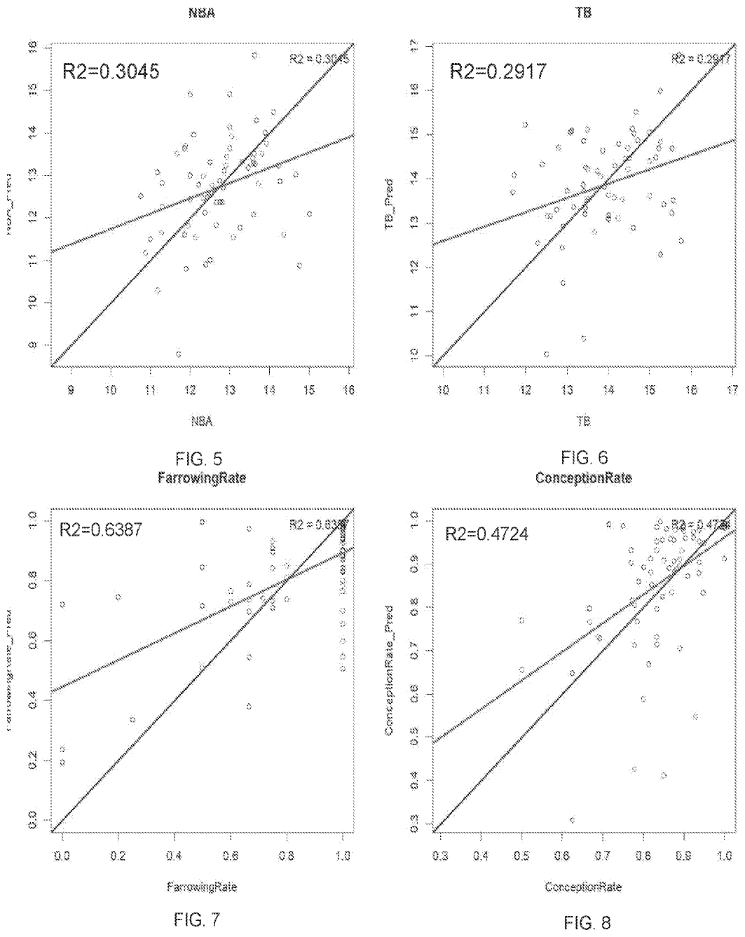

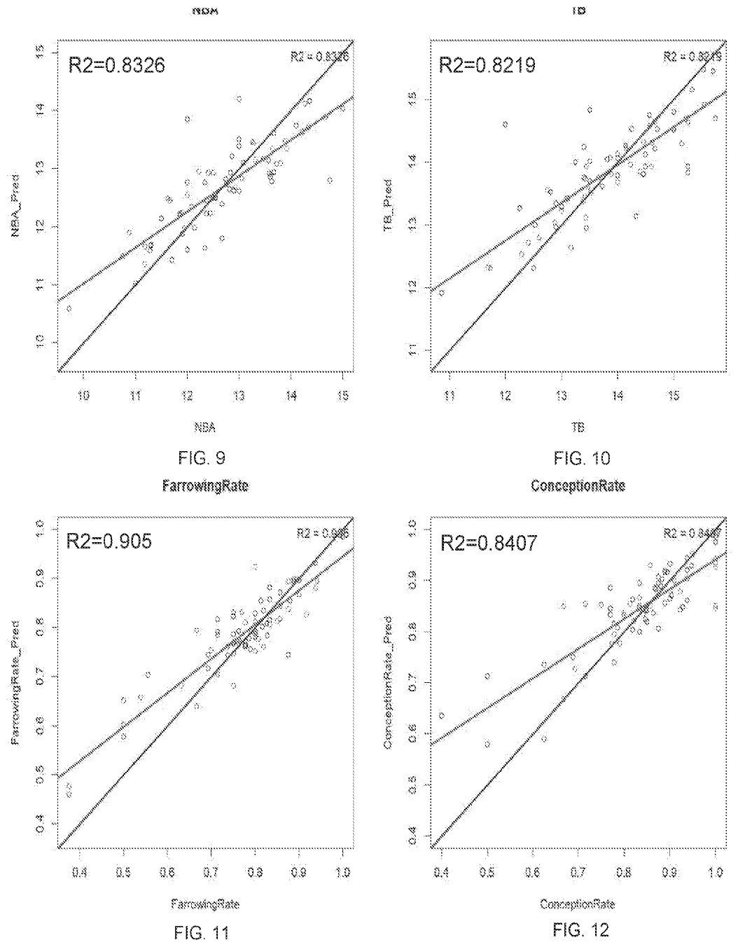

This may be visualized in FIGS. 1-12 where the predictive ability of the number of piglets to be born alive (“TBA”) ranges from an r² value of 0.84 (or 84%) for Combination 1 to 0.30 and 0.83 for Combinations 2 and 3, respectively. As mentioned above, the r² value may be a statistical measure (perhaps also known as a coefficient of determination) that represents the proportion of the variance of a dependent variable that is explained by an independent variable or variables in a regression model. Further, these data may be expressed and compared using a combination of r² and even an angle of the line in comparison to a perfect line which has an angle of 45°. These two values may be utilized independently or even used in combination perhaps to predict fertility-related parameters. As but one non-limiting example, a particular combination may have an r² value of 0.92 and an angle of 41.40 with a combined score of 0.855. The r² and/or the angle may be weighted such one or the other is valued as more predictive, or more important in assessing accuracy of the prediction relative to the perfect prediction. Each of the broad parameters may have sub-categories that are analyzed in one or more ways, using multiple types of laboratory equipment that may report similar, but perhaps slightly nuanced data that can impact the ability to predict fertility. In addition, within a single dataset, multiple pieces of data may be derived and reported such as the inverse of a particular data piece. As but one non-limiting example, both the positive (percentage intact DNA) and the negative (percentage of DNA having breaks) may both be utilized for data input.

In embodiments of the present invention, data input and even data output may be different for the different types of gamete, storage type, species, uses, or the like. As but one non-limiting example, semen that is frozen may be utilized to fertilize a freshly collected oocyte and may require at the least, input regarding the sperm quality attributes. As but another non-limiting example, frozen semen may be use to artificially inseminate a female, or fresh semen may be used to artificially inseminate a female and this may require oocyte, sperm quality, environmental data, or the like for accurate predictions. In yet another non-limiting example, cooled sperm may be utilized to fertilize an in vitro oocyte which may require sperm quality, oocyte quality, media, and even technician and environmental data to obtain an accurate, relative prediction on full-term pregnancy possibilities. Another non-limiting example may include the insemination of a superovulated female using cooled semen which may require information on sperm quality, environmental, and even technician data. In each case, it may be useful to understand the predicted fertility of the in vitro gamete such that one might have a better idea of the chance of success when using the particular gamete.

Success might be defined as conception, embryo production, pregnancy retention, farrowing, calving, foaling or similar parturition events, the chance of a live offspring, the number of offspring produced as a result of the insemination event, or the like. In a non-limiting example, a group of 130 boars may require culling to 100 total due to space limitations. This culling may utilize a combination of sperm quality and even environmental data to predict fertility. From the fertility data, a decision to cull 30 animals could be made. Alternatively, a producer may utilize a weighted set of fertility data, environmental data, perhaps combined with, or in addition to, genetic data to decide which animals should be culled.

In some embodiments, animal and gross gamete information may include, but is not limited to: identifying information for the particular individual; male weight at collection; nutritional status of the animal(s); age(s); testosterone level; body condition score; breeding schedule; parity; collection schedule; last collection date; gestation length; service number; matings in service; last parturition data; ease of parturition; complications at parturition; days since last pregnancy or days since last breeding; transportation or similar movement related stresses which may be measured by accelerometers, compass, gyroscopes, cortisol levels; and other information that may define the physical characteristics of the animals being used; or the like. Information on the gross characteristics of the sample collected may also be utilized and may include, but is not limited to: method of collection; concentration of the cell; concentration of groups of cells, concentrations of sperm; seminal plasma characteristics; follicular fluid characteristics; electrical conductivity of fluid; electrical conductivity of extended sperm; quality of the seminal plasma; storage period; storage temperature; refractometry; thermoresistance; type of media used to dilute samples; flushing characteristics; flushing number quality and grade of oocytes; flushing temperature; time between removal from an in vivo state to an in vitro state to a stable in vitro state such as cryopreservation or a second in vivo state (as may be explained by collection, cooling, transportation and insemination, or the like); flushing solutions; composition of such media; interaction of such media with said cells; or the like. Different kinds of animals may be evaluated such as but not limited to bovine, equine, ovine, porcine, caprine, avian, human, or the like.

Additional information may be evaluated such as but not limited to: the various collection methods utilized to obtain gametes; results from a biopsy; oxygen saturation of the media; quality of the water used in preparation of the solutions utilized for in vitro holding; the presence of metal ions in the water or media; the medium for maturation; fertilization or even culture of gametes, embryos; cooling information; cryopreservation information; or the like. The identification of bacterial, viral or even fungal contamination; presence of a biofilm; genus and species of such contamination may also be included.

In embodiments, the present invention may utilize semen qualities in predictions. Semen qualities may include, but are not limited to, semen state, animal information of said male animal, gross sperm information, morphological aspects, cellular motion, cellular function, regulation of intracellular information, reduction-oxidation balance, population of cells, sperm developmental conditions, personnel skills, any combination thereof, or the like. A semen state may be fresh sperm, frozen sperm, cooled sperm, vitrified sperm, sperm in gel state, thawed sperm, extended sperm, extended spermatozoa, extended semen, warmed sperm, freeze dried sperm, dehydrated sperm, rehydrated sperm, in vitro sperm, epidydimal sperm, multiple sperm cells, singular sperm cells, or the like. Animal information may be information such as identification of a particular animal (e.g., animal ID: number, other identifying characteristics), testosterone level, weight, nutritional status, genetic line, body condition score, breeding soundness exam, transportation, stress of a male animal, hemoglobin, fibronectin, inflammation markers, method of collection of sperm, electrical conductivity of sperm, presence of metal ions, concentration of sperm, hormone levels, temperature, any combination thereof, or the like. Gross sperm information may include seminal plasma information, electrical conductivity of fluid, storage period, storage temperature, refractometry, thermoresistance, type of solutions, solution characteristics, contaminants, contaminant concentration, organism contaminating, any combination thereof, or the like. Morphological aspects may include physical characteristics of cells, percentage normal cells, percentage abnormal cells, shape descriptions, volume, aspect ratios, ratios of physical sperm attributes to one another or between samples, total mass, concentration of sperm relative to seminal plasma, length, width, area, thickness, midpiece defects, abnormal heads, distal midpiece reflex, presence or absence of head, midpiece and tail, presence of all essential sperm parts, sperm intactness, any combination thereof, or the like. Cellular motion may include total motility, progressive motility, velocity descriptors, rate of motility, velocity of motility, percentage of cells in each velocity category, kinematic parameters, mean, median and mode of kinematic parameters, agglutination, any combination thereof, or the like. Cellular function may include acrosome quality, membrane quality, membrane fluidity, mitochondrial quality and depolarization, presence of aggresomes, ubiquitin, ubiquitinated proteins, zinc, zinc concentration, apoptotic cells, DNA quality, reciprocal translocations, single nucleotide polymorphism (SNP), seminal plasma proteins, data distribution differences, mitochondrial depolarization, and combination thereof, or the like. Regulation of intracellular information may include cAMP, MTOR-pathways, mineral composition, metal, zinc, calcium, cortisol, serotonin, hippocampal glucocorticoid, any combination thereof, or the like. Reduction-oxidation balance may include total antioxidant capacity of cells, total antioxidant capacity of extender, identification of an extender, total antioxidant capacity of seminal plasma, total antioxidant capacity intracellular, total antioxidant capacity extracellular, superoxide dismutase concentration, endogenous and exogenous antioxidants, presence of oxidants intracellular, presence of oxidants extracellular, presence of antioxidants intracellular, presence of antioxidants extracellular, membrane reduction-oxidation balance, oxidative damage, reactive oxygen species, reactive sulfur species, reactive nitrogen species, any combination thereof, or the like. Information about population of cells may include intensity of fluorescence of membrane, intensity of fluorescence of DNA, intensity of fluorescence of acrosomes, intensity of fluorescence of membrane fluidity, delta between fluorescent populations, median of fluorescence intensity for a specific population, mode of fluorescence intensity for a specific population, any combination thereof, or the like. Sperm developmental conditions may include temperature during spermatogenesis, temperature during spermiogenesis, humidity during spermatogenesis, humidity during spermiogenesis, temperature during collection, temperature during insemination, temperature of solutions, barometric conditions, barometric pressure, temperature of the testicles, any combination thereof, or the like. Personnel skills may include a technician's skills when thawing semen, a technician's skills when handling semen, a technician's skills when warming semen, a technician's skills when inseminating semen, and any combination thereof.

Embodiment of the present invention may utilize female characteristics in predictions. Predicting fertility of a female animal may include data that is not from a male animal in some embodiments. Female characteristics may include but are not limited to oocyte type, animal information of a female animal, gross gamete information, morphological aspects, cellular function, regulation of intracellular information, reduction-oxidation balance, population of cells, oocyte developmental conditions, personnel skills, any combination thereof, or the like. An oocyte type may include fresh oocytes, frozen oocytes, cooled oocytes, superovulated oocytes, flushed oocyte, vitrified oocyte, thawed oocyte, freeze dried oocytes, warmed oocyte, donor oocyte, in vivo oocyte, in vitro oocyte, in utero oocyte, any combination thereof, or the like. Animal information may include weight, nutritional status, body condition score, parturition information, weaning information, breeding schedule, parity, body temperature, vulvar temperature, uterine temperature, transportation, stress of a female animal, synchronization protocol, hemoglobin, fibronectin, inflammation markers, method of collection of oocytes, hormone levels, uterine health, reproductive health, involution state of uterus, follicle health, follicle maturity, donor information, recipient information, temperature, any combination thereof, or the like. Gross gamete information may include follicular fluid, electrical conductivity of fluid, storage period, storage temperature, refractometry, thermoresistance, type of solutions, solution characteristics, oxygen saturation of solutions, contaminants, synchronization protocol, follicle health, follicle maturity, oocyte health, any combination thereof, or the like. Morphological aspects may include physical characteristics of oocyte cells from a female animal, shape descriptions of oocytes from a female animal, volume of oocytes from a female animal, ratios of cellular dimensions, distributions, compactness, total mass of oocytes from a female animal, cumulus cell to oocyte mass, number of oocytes/dish, spindle formation, chromosome location and position, mitochondrial distribution, organelle distribution, percentage of normal oocytes from a female animal, length of oocytes from a female animal, width of oocytes from a female animal, area of oocytes from a female animal, any combination thereof, or the like Cellular function may include information about oocytes from a female animal such as but not limited to membrane quality, membrane fluidity, aggresomes, ubiquitin, ubiquitinated proteins, zinc concentration, apoptotic cells, spindle formation, DNA quality, reciprocal translocations, cellular division status, oviductal cell binding, single nucleotide polymorphism (SNP), organelle distribution, data distribution differences, mitochondrial depolarization, any combination thereof, or the like. Regulation of intracellular information may include cAMP, MTOR-pathways, mineral composition, metal, zinc, calcium, cortisol, serotonin, hippocampal glucocorticoid, presence of ubiquitin, presence of zinc, cell surface markers, any combination thereof, or the like. Reduction-oxidative may include total antioxidant capacity of cells, total antioxidant capacity of fluids, total antioxidant capacity intracellular, total antioxidant capacity extracellular, intracellular antioxidants, extracellular antioxidants, presence of oxidants intracellular, presence of oxidants extracellular, membrane reduction oxidative balance, presence of CD9, oxidative damage, reactive oxygen species, presence of reactive nitrogen species, presence of reactive sulfur species, any combination thereof, or the like. Population of cells may include information about oocytes from a female animal such as but not limited to intensity of fluorescence of membrane, intensity of fluorescence of DNA, intensity of fluorescence of acrosomes, intensity of fluorescence of membrane fluidity, delta between fluorescent populations, average fluorescence intensity between a group of cells, average fluorescence intensity of individual cells, any combination thereof, or the like. Oocyte developmental conditions may include temperature during oogenesis, humidity during oogenesis, temperature during oocyte collection, temperature during oocyte culture, humidity during oocyte collection and culture, a change in temperature, the timeline between critical events within the process, temperature during insemination, temperature during culture, temperature of solutions, barometric conditions, any combination thereof, or the like. Personnel skills may include a technician's skills when thawing oocytes from a female animal, a technician's skills when handling oocytes from a female animal, a technician's skills when warming oocytes from a female animal, a technician's skills when implanting, a technician's skills when culturing, any combination thereof, or the like Fertility-related parameters perhaps for a female animal may include farrowing rate, kidding rate, oocyte quality, oocyte fertility potential, oocyte health, post-thaw oocyte health, calving rate, foaling rate, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, any combination thereof, or the like.

Embodiment of the present invention may utilize embryo characteristics in predictions. Embryo characteristics may include but are not limited to gamete type, animal information, gross gamete information, morphological aspects, cellular function, regulation of intracellular information, reduction-oxidation balance, population of cells, gamete developmental conditions, personnel skills, any combination thereof, or the like. Gamete type may include fresh, frozen, cooled, flushed embryo, superovulated oocytes, flushed oocyte, any combination thereof, or the like. Animal information may include weight, nutritional status, body condition score, parturition information, breeding schedule, parity, transportation, stress of an animal, synchronization protocol, hemoglobin, fibronectin, inflammation markers, method of collection, hormone levels, donor information, recipient information, temperature, any combination thereof, or the like. Gross gamete information from gametes used to create an embryo may include follicular fluid, electrical conductivity of fluid, storage period, storage temperature, refractometry, thermoresistance, type of solutions, solution characteristics, contaminants, synchronization protocol, follicle health, follicle maturity, oocyte health, any combination thereof, or the like. In some embodiments an embryo may be evaluated for a variety of traits when it is in a cleavage stage, a blastocyst stage or even a hatching stage. Gross information may also include location of implantation, estrus status of the recipient, correlation of estrus status and embryo state. Morphological aspects may include physical characteristics of an embryo, grade of embryo, shape descriptions of an embryo, volume of an embryo, ratios of physical attributes to one another, total mass of an embryo, mitochondrial distribution, organelle distribution, percentage of normal cells, length of said embryo, width of an embryo, area of an embryo, any combination thereof, or the like. Cellular function may include information about an embryo such as but not limited to membrane quality, membrane fluidity, aggresomes, ubiquitin, ubiquitinated proteins, zinc concentration, apoptotic cells, spindle formation, DNA quality, reciprocal translocations, cellular division status, oviductal cell binding, single nucleotide polymorphism (SNP), organelle distribution, data distribution differences, mitochondrial depolarization, any combination thereof, or the like. Regulation of intracellular information may include cAMP, MTOR-pathways, mineral composition, metal, zinc, calcium, cortisol, serotonin, hippocampal glucocorticoid, any combination thereof, or the like. Reduction-oxidation balance may include total antioxidant capacity of cells, total antioxidant capacity of the embryo, total antioxidant capacity of fluids, total antioxidant capacity intracellular, total antioxidant capacity extracellular, presence of oxidants intracellular, presence of oxidants extracellular, membrane reduction oxidative balance, oxidative damage, reactive oxygen species, presence of CD9, oxidative damage, reactive oxygen species, presence of reactive nitrogen species, presence of reactive sulfur species, any combination thereof, or the like. Population of cells may include information about an embryo such as intensity of fluorescence of membrane, intensity of fluorescence of DNA, intensity of fluorescence of acrosomes, intensity of fluorescence of membrane fluidity, delta between fluorescent populations, any combination thereof, or the like. Gamete developmental conditions may include temperature during oogenesis, humidity during oogenesis, temperature during insemination, temperature during culture, temperature of solutions, barometric conditions, any combination thereof, or the like. Personnel skills may include a technician's skills when thawing an embryo, a technician's skills when handling an embryo, a technician's skills when warming an embryo, a technician's skills when implanting an embryo, any combination thereof, or the like. An embryo success rate may include a parameter such as implantation chance, 25-35-, 45-, or perhaps even 60-day pregnancy detection, presence of pregnancy related hormones, pregnancy retention, parturition rate, total number of animals born alive, total number of animals born, number of mummified fetuses, health of the offspring, physical normality of the offspring or the like. An embryo success rate may include a parameter such as but not limited to calving rate, foaling rate, farrowing rate, kidding rate, development of embryo, embryo quality, post-thaw embryo health, embryo transplant success, embryo transfer success, superovulation embryo transfer success, fecundity, fecundability, any combination thereof, and the like.

Embodiment of the present invention may utilize qualities from both a male and female animal in predictions. Such qualities may include but are not limited to gamete type, animal information, gross gamete information, morphological aspects, cellular motion, cellular function, regulation of intracellular information, reduction-oxidation balance, population of cells, gamete developmental conditions, personnel skills, any combination thereof, or the like. Gamete type may include fresh, frozen, cooled, flushed embryo, retrieved oocyte, superovulated oocyte, flushed oocyte, fresh sperm, frozen sperm, cooled sperm, vitrified sperm, sperm in gel state, thawed sperm, extended sperm, warmed sperm, freeze dried sperm, dehydrated sperm, rehydrated sperm, in vitro sperm, epidydimal sperm, multiple sperm cells, singular sperm cells, extended semen, any combination thereof, or the like. Animal information may include weight, nutritional status, body condition score, parturition information, breeding schedule, parity, transportation, stress of an animal, synchronization protocol, hemoglobin, fibronectin, inflammation markers, method of collection of oocytes, concentration, hormone levels, donor information, recipient information, temperature, any combination thereof, or the like. Gross gamete information may include seminal plasma information, follicular fluid, electrical conductivity of fluid, storage period, storage temperature, refractometry, thermoresistance, type of solutions, solution characteristics, contaminants, contaminant concentration, organism contaminating, synchronization protocol, follicle health, follicle maturity, oocyte health, any combination thereof, or the like. Morphological aspects may include physical characteristics of cells, percentage abnormal cells, shape descriptions, volume, aspect ratios, ratios of attributes to one another, total mass, mitochondrial distribution, organelle distribution, concentration relative to seminal plasma, percentage of normal cells, length, width, area, thickness, midpiece defects, abnormal heads, distal midpiece reflex, any combination thereof, or the like. Cellular motion may include total motility, progressive motility, velocity descriptors, rate of motility, velocity of motility, percentage of cells in each velocity category, kinematic parameters, mean, median and mode of kinematic parameters, agglutination, any combination thereof, or the like. Cellular function may include acrosome quality, membrane quality, membrane fluidity, mitochondrial quality and depolarization, presence of aggresomes, ubiquitin, ubiquitinated proteins, zinc, zinc concentration, apoptotic cells, spindle formation, DNA quality, reciprocal translocations, cellular division status, oviductal cell binding, single nucleotide polymorphism (SNP), seminal plasma proteins, organelle distribution, data distribution differences, mitochondrial depolarization, any combination thereof, or the like. Regulation of intracellular information may include cAMP, MTOR-pathways, mineral composition, metal, zinc, calcium, cortisol, serotonin, hippocampal glucocorticoid, any combination thereof, or the like. Reduction-oxidation balance may include total antioxidant capacity of cells, total antioxidant capacity of fluids, total antioxidant capacity intracellular, total antioxidant capacity extracellular, presence of oxidants intracellular, presence of oxidants extracellular, membrane reduction oxidative balance, oxidative damage, reactive oxygen species, any combination thereof, or the like. Population of cells may include intensity of fluorescence of membrane, intensity of fluorescence of DNA, intensity of fluorescence of acrosomes, intensity of fluorescence of membrane fluidity, delta between fluorescent populations, median of fluorescence intensity for a specific population, mode of fluorescence intensity for a specific population, any combination thereof, or the like. Developmental conditions may include temperature during spermatogenesis, temperature during spermiogenesis, humidity during spermatogenesis, humidity during spermiogenesis, temperature during oogenesis, humidity during oogenesis, temperature during insemination, temperature during culture, temperature of solutions, barometric conditions, any combination thereof, or the like. Personnel skills may include a technician's skills when thawing cells, a technician's skills when handling cells, a technician's skills when warming cells, a technician's skills when inseminating cells, a technician's skills when implanting cells, any combination thereof, or the like.

Embodiments of the present invention may utilize intracellular or even extracellular qualities of a reproductive cell in predictions. Reproductive cells may include but is not limited to stem cells, pluripotent stem cells, gametes, sperm cells, oocytes, embryos, haploid cells, spermatozoon, ovum, somatic cell nuclear transfer results, small cell nuclear transfer results, parthenogenic embryos, or the like. Intracellular or extracellular qualities are chosen from gamete type, animal information, gross gamete information, morphological aspects, cellular function, regulation of intracellular information, reduction-oxidation balance, population of cells, developmental conditions, personnel skills, any combination thereof, or the like. Gamete type may include fresh, frozen, cooled, flushed embryo, retrieved oocyte, superovulated oocyte, flushed oocyte, fresh sperm, frozen sperm, cooled sperm, vitrified sperm, sperm in gel state, thawed sperm, extended sperm, warmed sperm, freeze dried sperm, dehydrated sperm, rehydrated sperm, in vitro sperm, epidydimal sperm, multiple sperm cells, singular sperm cells, any combination thereof, or the like. Animal information may include weight, nutritional status, body condition score, parturition information, breeding schedule, parity, transportation, stress of said animal, synchronization protocol, hemoglobin, fibronectin, inflammation markers, method of collection of oocytes, concentration, hormone levels, donor information, recipient information, temperature, any combination thereof, or the like. Gross gamete information may include seminal plasma information, follicular fluid, electrical conductivity of fluid, storage period, storage temperature, refractometry, thermoresistance, type of solutions, solution characteristics, contaminants, contaminant concentration, organism contaminating, synchronization protocol, follicle health, follicle maturity, oocyte health, any combination thereof, or the like. Morphological aspects may include physical characteristics of cells, percentage abnormal cells, shape descriptions, volume, aspect ratios, ratios of attributes to one another, total mass, mitochondrial distribution, organelle distribution, concentration relative to seminal plasma, percentage of normal cells, length, width, area, thickness, midpiece defects, abnormal heads, distal midpiece reflex, any combination thereof, or the like. Cellular motion may include total motility, progressive motility, velocity descriptors, rate of motility, velocity of motility, percentage of cells in each velocity category, kinematic parameters, mean, median and mode of kinematic parameters, agglutination, any combination thereof, or the like. Cellular function may include acrosome quality, membrane quality, membrane fluidity, mitochondrial quality and depolarization, presence of aggresomes, ubiquitin, ubiquitinated proteins, zinc, zinc concentration, apoptotic cells, spindle formation, DNA quality, reciprocal translocations, cellular division status, oviductal cell binding, single nucleotide polymorphism (SNP), seminal plasma proteins, organelle distribution, data distribution differences, mitochondrial depolarization, any combination thereof, or the like. Regulation of intracellular information may include cAMP, MTOR-pathways, mineral composition, metal, zinc, calcium, cortisol, serotonin, hippocampal glucocorticoid, any combination thereof, or the like. Reduction-oxidation balance may include total antioxidant capacity of cells, total antioxidant capacity of fluids, total antioxidant capacity intracellular, total antioxidant capacity extracellular, presence of oxidants intracellular, presence of oxidants extracellular, membrane reduction oxidative balance, oxidative damage, reactive oxygen species, any combination thereof, or the like. Population of cells may include intensity of fluorescence of membrane, intensity of fluorescence of DNA, intensity of fluorescence of acrosomes, intensity of fluorescence of membrane fluidity, delta between fluorescent populations, median of fluorescence intensity for a specific population, mode of fluorescence intensity for a specific population, any combination thereof, or the like. Developmental conditions may include temperature during spermatogenesis, temperature during spermiogenesis, humidity during spermatogenesis, humidity during spermiogenesis, temperature during oogenesis, humidity during oogenesis, temperature during insemination, temperature during culture, temperature of solutions, barometric conditions, any combination thereof, or the like. Personnel skills may include a technician's skills when thawing cells, a technician's skills when handling cells, a technician's skills when warming cells, a technician's skills when inseminating cells, a technician's skills when implanting cells, any combination thereof, or the like.

For any of the embodiments discussed herein, a prediction may be accomplished perhaps based on only one quality, characteristic, or the like and in other embodiments predictions can be based on more than one quality, characteristic, or the like, perhaps using a prediction models automated computational transformation algorithm.

Embodiments of the present invention may utilize inputs, data, attributes, values, responses or even characteristics which may include direct female related parameters such as, but not limited to: female body weight; body condition score; synchronization protocol utilized; follicle health; follicle maturity; oocyte health; DNA fragmentation within an oocyte; hemoglobin; fibronectin; inflammation markers such as cortisol, follicle stimulating hormone levels, progesterone, luteinizing hormone level, or the like; oocyte surface markers; mitochondrial health; mitochondrial distribution; or the like. Other items may include, but are not limited to: an average temperature of the female; the temperature of the female at ovulation; the temperature of the female at insemination; the delta of the environmental temperature and even the body temperature of the animal at various critical time points; a combination of temperatures of the gametes, temperature of the testicles, the body, the uterine environment; or the like. Inputs, data, attributes, values, responses or characteristics may additionally include embryo measurements perhaps immediately after collection from a donor or even immediately before implantation.

In other embodiments, the present invention may use information as inputs, data, or the like which may include, but is not limited to: which fluids contact and even impact cellular functions; cellular body fluids; seminal plasma characteristics; ejaculate color; percentage of seminal plasma relative to sperm concentration; or the like. These may include, but are not limited to cumulus cells; follicular fluid; other cellular in vivo media or cytoplasmic solutions; or the like. In addition, this may include various media that may be utilized to protect cells, mature cells, preserve cells, cryopreserve cells, cool cells, flush cells, or the like. It may also include, in some embodiments, components of such contacting fluids including but not limited to: concentration of zinc; concentration of heavy metals, concentration of oxygen, presence of superoxide dismutase; presence of antioxidants; reactive and charged species; moieties that may impact cellular integrity, functionality, and perhaps even capacity to perform to full competence.

In some embodiments, inputs, data, or the like may include physical cellular aspects or even morphological traits such as descriptions of the physical characteristics of the cell, cells or multicellular composite, or the like. This may include, but is not limited to; aspect ratios; angle; shape rations; definition of different axes of the cell such as length, width, diameter, thickness, density; cell volume; or the like. Each of these may be represented as: pixels; intensity; gradient of such density, pixels or intensity: compactness; luminescence, fluorescence, or eve uniformity of a signal; or the like. It may include ratios of physical components of cells such as head to total length; tail to total length; length of midpiece; midpiece relative to other sperm portions; cumulus cells to oocyte mass; or the like. Total mass, weight, and even density may be included in physical characteristics. Inputs, data, or the like may broadly include: cellular concentration perhaps as individual cells relative to concentration of seminal fluid: number of oocytes in a dish; distribution of mitochondria or other organelles within an oocyte, cumulus cells or embryo; or the like. Physical descriptors may include, but is not limited to: percentage of normal cells; percentage of abnormal cells; percentage of detached heads; percentage of abnormal tails; percentage of midpiece defects; percentage of distal midpiece reflexes; percentage of abnormally shaped heads; sperm length; width; area; a mathematical combination of sperm length, width and area; or the like.

Embodiments describing cellular motion such as motility may include, but is not limited to: the percentage of cells that are moving; the percentage of cells that are moving correctly or perhaps progressively; rate of velocity; hypermotility; flagellar movement; the average, mean, median or even mode of motile cells; velocity parameters; straightness; or the like. Kinematic parameters such as those that may be described by a computer assisted sperm analyzer or other such technology that can analyze motion of a cell relative to a standard may also be included in this category. These may include, but is not limited to: average path velocity; percentage of slow, medium or rapid swimming cells; straight line velocity; beat frequency; curvilinear velocity; the numbers of cells in each category; the straightness of cellular movement; the velocity of cellular movement; sperm head displacement; flagellar movement; or the like as well as mean, median and mode of such; or the like. The percentage of a given population of cells, number or absolute number of cells falling into certain groupings such as slow, medium or rapid may also be utilized. Measurements may include a percentage of aggregated, agglutinated, clumped or even tangled cells, or the like.

In yet other embodiments, cellular functional parameters may include, but are not limited to: quality of acrosome, membrane, membrane fluidity, membrane stability, DNA, mitochondria, or the like; membrane fluidity; presence of aggresomes; presence of ubiquitin and even ubiquitinated proteins; presence of zinc; location of zinc, ubiquinated proteins or the like; relative concentration of zinc, ubiquinated proteins or the like within a cell; apoptotic cells; spindle formation; DNA status regarding cellular divisions; DNA breaks; presence of single or even double stranded breaks; or the like. These may include functionality measurements such as oviductal cell binding assays; single nucleotide polymorphism profile; or perhaps even the presence of various seminal plasma proteins or cell surface proteins. Other cellular functionality measurements may include organellular distribution within a cell or grouping of cells including but not limited to: mitochondrial arrangement; mitochondrial charge; depolarization; polarization; sheath length; endoplasmic reticulum arrangement; smooth endoplasmic reticulum arrangement golgi body arrangement; lysosomal arrangement individually, in relation to one another or even in relation to some other point within the organism or cell; or the like. Relative volumes occupied by organelles in specific regions such as the cortex, subcortex, inner cytoplasm, or the like may also be utilized as predictive data.

The data describing the cellular functionality may include but is not limited to: differences between representative data points; graphically distributed datapoints; microscopically visualized datapoints; data spots and/or between different types of data visualization; or the like. This may include, but is not limited to: distance between two spots; median fluorescence; mode fluorescence; mean fluorescence, perhaps within or even between population(s); ratios between different parameters; relative fluorescence intensity; absolute fluorescence intensity; comparison of fluorescence intensity between and among populations; or similar measurements or data; or the like. In addition, distance between multiple peaks, a total peak area, a peak area that overlaps, a comparison of peak areas, a high or low intensity peak mean fluorescence, or the like, may be utilized to represent cellular characteristics. Such parameters may include, but are not limited to: measurements of individual cells, groups of cells, or composite of cells or may even be aggregate data of hundreds, or thousands of cells or measurements. As a non-limiting example, 10,000 sperm cells may be measured and a subset (e.g., 50%) of these 10,000 cells may have intact acrosomes (5000 cells) and 30% of the acrosome intact cells may have DNA with no breaks (1,500 or 15% of the original cells). These percentages as a whole, separately and/or in aggregate may be utilized for data input. Additionally, these may include multiple measures for cells, such as acrosome intact plus membrane intact or acrosome compromised/reacted plus leaky membranes (sometimes referred to as dead and reacted). Similarly, mitochondria may be defined as depolarized or “mitochondria positive” and the mean and median of the data output describing “mitochondria positive” may be utilized. The aforementioned may be assessed by a variety of stains, dyes, and/or visualization methods and it should be understood that data without regard to method of collection, may be an important factor in assessment. Assessment of these parameters may also include cells that are in a transition state. As but one non-limiting example, cells may be membrane stable shifting to unstable. A percentage or even an absolute number of cells within a population may be important to define.

In other embodiments, data, input, or the like may include the presence of ubiquitin, or even ubiquitinated proteins in certain locations within a cell. A fluorescence intensity of ubiquitinated proteins and location within the cell may be characterized and may even be numerically defined and this value(s) may be used to analyze fertility or similar parameters for breeding or culling decisions. Other apoptotic markers may serve as inputs, data, attributes, values, responses or even characteristics for either male or female related parameters or within sperm, oocytes or embryos.

Information on the molecular function of cells may include, but is not limited to, the regulation of intracellular cAMP signaling pathway; mechanistic target of rapamycin (MTOR-specific pathways); cellular integrity as measured by these pathways; mineral or even metal composition within a cell or exterior to a cell; presence of zinc within a cell or being excreted by a cell: zinc binding proteins; location of zinc binding proteins within a cell; zinc homeostasis; changes in zinc localization over a cellular event; zinc signatures; total zinc concentration; zinc content of the epididymis; testes dorsolateral prostate or even enzyme activity as affected by zinc intracellular calcium; or the like.

Cellular markers for stress may ultimately impact fertility or transplant success may be measured. These may include, but are not limited to: cortisol; serotonin; serotonin responses; corticostriatal neuron action; hippocampal glucocorticoid receptor expression; genetic markers of stress; or the like. Other information may include analysis of healthy cells, groups of cells or even cellular aggregation such as an embryo. A more in-depth analysis of cells that are considered by other measures to be healthy may be applied.

Embodiments of the present invention may include data, input, or the like relating to the balance of oxidants and antioxidants perhaps both intracellular and extracellular. This may include, but is not limited to: total antioxidant capacity of seminal plasma; of solutions surrounding the cells; of medium in which cells are incubated including redox activity, antiradical activity; or the like. Such may be measured by assays including antiradical activity, ORAC, FRAP, TRAP, TEAC, TAR, AFAX, DPPH, EC50 and other similar data expressions and methods; or the like. These methods may be utilized to assess an in vitro and/or in vivo production of antioxidants, antioxidants in the environment surrounding the cells, cellular aggregates or tissues including measures of in vitro and in vivo oxidative stress, measure of free radicals; or the like. This may include direct or even indirect measures of concentrations of: reactive oxygen species; reactive nitrogen species; reactive sulfur species; oxygen; superoxide anion; superoxide radical; hydroperoxyl radical; hydrogen peroxide; hydroxyl radical; reactive nitrogen species such as but not limited to peroxynitrite, alkyl peroxynitrites, dinitrogen trioxide, dinitrogen tetroxide, nitrous acid, nitroium anion, nitroxyl anion, nitric oxide, nitrogen dioxide, nitrosyl cation, nitryl chloride; reactive sulfur species such as but not limited to mixed disulfide, perthiyl radical, perthiolate, persulfides, polysulfides, thiosulfate; or the like. Data may include measurements of even antioxidants such as but not limited to catalase, superoxide dismutase and glutathione, coenzyme Q10, lipoic acid, butylated hydroxytoluene; or the like. Exposure to oxidants, antioxidant level, oxidation level of membranes or associated fluids, membrane lipid oxidation level, oxidants in the seminal plasma, superoxide dismutase levels, markers on the surface of the cell, proteins on the surface of the cell, the presence or absence of CD9, or the like may be information to include in fertility projections. In addition, factors that may be understood to induce oxidative damage, reduce the effectiveness of antioxidants such as ambient, UV or LED light exposure, quality of water, presence of iron or other reactive metals, quality of chemicals utilized in preparation of media that may contain oxidants, or even antioxidants, as well as the relative concentration of oxidants in solution may be quantified and may be utilized in evaluating fertility projections.

Embodiments of the present invention may utilize attributes of individual populations or even subpopulations of cells that might prove more predictive for culling and/or even breeding accuracy. As but one nonlimiting example, the intensity of fluorescence emitted by sperm cells having intact acrosomes may be further analyzed to include all those having fully intact acrosomes but slightly less fluorescence intensity. Said fluorescence intensity of a population may be deemed to be healthy and fluorescence intensity of another population may be deemed to be healthy but less so. This may be analyzed by the delta between these two populations, the median, mode or mean of said fluorescence characteristics or even the difference in fluorescence characteristics between two different time points, two or more different, distinct populations, a series of populations, or the like.

In other embodiments, cellular and/or gamete developmental conditions may include temperatures, humidity, or the like perhaps at critical junctures in development of these cells including but not limited to: temperatures at oogenesis; spermatogenesis; spermiogenesis; release of oocyte; temperature between about 1 and about 55 days prior to collection; humidity between about 1 and about 55 days prior to collection; relative humidity between about 1 and about 55 days prior to collection; the average high temperatures for such days; the average humidity for such days; or the like. Other data may include the sum of temperatures and humidity scores for a critical series of days, temperature and humidity at the time of collection, temperature of the sample at processing, temperature of the sample pre and post-transport, or the like. It may include the difference in temperature, the change of temperature, the net change within a given period of time or between critical events, and even the oscillations of temperature within a given critical set of events, or the like. Other sets of input data may include environmental conditions and mating information which may include the temperature, humidity, relative humidity, relative temperature at time of insemination, time of collection, time of significant events, or the like. Moreover, this may include date, time of year, location of insemination, location of implantation, location of retrieval, sperm temperature, process of sperm dilution (addition of sperm to extender or extender to raw sperm), processing time, steps in processing, temperature, temperature gradient, sedimentation, technician processing, temperature of samples at delivery, temperature delta between collection and use of oscillations relating to such and various datapoints relative to said measurements that may include the difference, the extremes, the time of such extremes the insulation or protection buffering against such extremes, or the like. Other data may include daily, hourly or some similar periodicity of environmental measurements such as but not limited to geographic considerations, distance, topographic variation, elevation changes, barometric pressure changes, absolute temperature, humidity of the ambient environment, temperature, humidity, relative temperature, relative humidity, ambient light, circadian cycles, or the like. A difference between the environmental temperature and the body temperature may be included. Each of the environmental measurements and factors may be included at the time of collection, the time of cellular production, at critical developmental points (e.g., such as spermatogenesis, spermiogenesis, or the like), the time of harvest from the body, in vitro use, or the like. Environmental samples may be conducted in the area relatively close to the location of collection or even within the location of collection. Any or all of the aforementioned attributes may be included in the analysis or as a predictive variable within a model.

In other embodiment, data may come from disparate sources, but may provide or even define similar characteristics that may be useful to assist in the optimization of fertility prediction. These may be necessary to standardize, optimize, or otherwise improve the predictive abilities and the data integrity across a variety of laboratories, farms, groups or the like that have a variety of capabilities, different equipment, different information gathering, different data output, different historical information, different reporting software, different types of herds, different types of herd composition, the like. Data may be somewhat duplicative but may be found to be important as one type of machinery may see subtle, yet important differences in the cell or even situation being analyzed. As but one non-limiting example, the quality of the acrosome may be assessed by a flow cytometer and also by a visual morphology assessment. The visual morphology assessment may provide some data on the acrosome quality such as the percentage of the acrosome that may be missing, the type of damage that was incurred, while flow cytometry data may give general information such as the percentage of the populations that may be damaged, identification of the populations that have a very small, visually unidentified, discrepancy in acrosome quality or the like. As but another non-limiting example, sublethal changes may be detected by a flow cytometer as a tertiary population while in a computer assisted sperm analyzer machine a slow population with a decreased average path velocity may be detected.

Predictive data may include the quality and skills of the person handling, thawing, warming, or the like of the cells, gametes or embryos. Additionally, all human interactions may be considered to be influential including but not limited to: the technician used to collect, freeze, cool, thaw or warm gametes or embryos; transportation of said cells; storage; the person or people doing the insemination or implantation; temperature of the environment; light intensity; arrival temperature; length at elevated temperature; or other calculations that may ascertain the percentage of damage, damage by increased temperature, method of sperm collection, etc.; or the like.

Attributes may include any or all of the following attributes as defined by machines such as but not limited to an image flow cytometer, flow cytometer, fluorescence activated cell sorter, computer assisted sperm analyzer, pH meter, osmometer, microscope, hemocytometer, fluorescent microscope, thermometer, incubator, polymerase chain reaction machine, spectrophotometer, NucleoCounter®, cell counters, luminometer, computer analysis of data, programs such as FlowJo™, combinations of such machines, equipment and programs, or the like some of which are included in Table 1. These may be defined by other types of equipment, stains, dyes, imaging methods, surface assessment, analysis that may measure attributes such as shape, length, width, three dimensional attributes, total length, height, or the like. Moreover, attributes that positively or even negative correlate with said attributes may also be utilized to supplement a model creation, model accuracy, and even predictive abilities. These attributes may directly or indirectly measure cellular attributes, or may, through reflection of light, angles of light refractation or reflection or even transparency, or the like.

As referenced in Table 1 below, attributes that may be included in a predictive analysis may include both traditional analysis by flow cytometry, microscopic analysis morphological analysis, cellular dimensions, physical descriptors or the like. Data may be derived from a variety of sources including an image-flow cytometer, a flow cytometer, a microscope, a computer assisted sperm analyzer, a bright field microscope, or the like. Non-limiting examples of attributes and descriptions thereof are included in Table 1.

TABLE 1

| Attribute | Description |
|---|---|
| Per_Viable_with_Intact_Acrosome | Percent of cells with intact acrosomes and |
| | intact membrane. |
| CompDNA | Percentage of compromised or broken DNA |
| | (double or single standard). |
| Count, Mid-length & Sperm w Tail (BF | Number of cells having an attached tail and |
| Defined) | of intermediate length. |
| Per_Depolarized_Mitochondria | Percentage of cells having depolarized |
| | mitochondria. |
| Minor Axis Intensity_M06_6-SSC | Fluorescence intensity of a particular axis of |
| | a cell. |
| Oxidation_Basal.Induced_Ratio | The ratio of oxidized moieties to the |
| | baseline level of oxidation to determine the |
| | redox balance of a cell, system, media, and |
| | medium. |
| Per_Mero_Negative | Percentage of cells that are not stained with |
| | merocyanine. |
| Symmetry 3_Head H Defined_7-H33342 | The head symmetry as defined by Hoechst |
| | 33342. |
| Angle Intensity_M06_6-SSC | Reflection of light from a cell. |
| Intensity_AGG, Whole_2-AGG, Mean, | The intensity of aggresome staining plus an |
| Intensity (all), - & Sperm w Tail (BF | attached sperm tail. |
| Defined) | |
| Midpiece Length v3.0, Mean, Length, All & | Length of the midpiece of a sperm cell. |
| Sperm w Tail (BF Defined) | Length of the midpiece relative to the |
| | distribution of all midpieces measured |
| | within all sperm cells having an attached |
| | tail. |
| Compactness_M06_6-SSC | A physical measure of the cell based on |
| | light scattering and distribution. |
| Count, Head: Whole AGG Value & Sperm w | Assessment of percentage of aggresomes in |
| Tail (BF Defined) | a cell based on bright field microscopy and |
| | parsed based on physical characteristics. |
| Bright Detail Intensity No AGG | Lack of aggresomes and cell analysis based |
| | on light intensity. |
| Circularity_M06 | A physical measure of the cell based on |
| | light scattering and distribution. |
| Month | Time of year. |
| Per_Normal_Morphology | Those cells having normal or average |
| | physical cellular characteristics. |
| Bright Detail Intensity Mid AGG | A middle concentration of aggresomes |
| | combined with physical measure of the cell |
| | based on light scattering and distribution. |
| Symmetry 3_M06_6-SSC | Physical measure of the cell based on light |
| | scattering and distribution. |
| Max Contour Position_Head H Defined_7- | Physical measure of the cell based on light |
| H33342 | scattering and distribution defined by a stain |
| | known as Hoechst 33342. |
| Valley Y_M06_6-SSC | Distance between two light distributions of |
| | a cell or group of cells |
| Diameter_Head H Defined | Physical measure of the cell based on light |
| | scattering and distribution. |
| Thickness Min_M06 | Physical measure of the cell based on light |
| | scattering and distribution. |
| H Energy Mean_M06_6-SSC_5 | Physical measure of the cell based on light |
| | scattering and distribution. |
| Major Axis Intensity_M06_6-SSC | Physical measure of the cell based on light |
| | scattering and distribution. |
| Aspect Ratio Intensity_Head H Defined_7- | Physical measure of the cell based on light |
| H33342 | scattering and distribution in comparison to |
| | another physical attribute defined by DNA |
| | identifying dye Hoechst 33342. |
| Symmetry 2_M06_6-SSC | Physical measure of the cell based on light |
| | scattering and distribution. |
| Symmetry 4_M06_6-SSC | Physical measure of the cell based on light |
| | scattering and distribution. |
| Intensity_AGG, Whole_2-AGG, Mean, | A concentration of aggresomes combined |
| Intensity (all), + & Sperm w Tail (BF | with physical measure of the cell based on |
| Defined) | light scattering and distribution and |
| | including those cells that are physically |
| | intact as defined by bright field microscopy. |
| Contrast_M06_6-SSC | Physical measure of the cell based on light |
| | scattering and distribution. |
| H Correlation Mean_M06_6-SSC_5 | Physical measure of the cell based on light |
| | scattering and distribution. |
| Intensity_AGG, Whole_2-AGG, Mean, | A concentration of aggresomes combined |
| Intensity (all), ++ & Sperm w Tail (BF | with physical measure of the cell based on |
| Defined) | light scattering and distribution and |
| | including those cells that are physically |
| | intact as defined by bright field microscopy. |
| Midpiece Length v3.0, Mean, Short & Sperm | Length of a specific portion of the cell that |
| w Tail (BF Defined) | is physically intact and with specific |
| | attributes as defined by bright field |
| | microscopy. |
| Aspect Ratio_M01 | Physical measure of the cell based on light |
| | scattering and distribution. |
| Symmetry 2_Head H Defined_7-H33342 | Physical measure of the cell based on light |
| | scattering and distribution in comparison to |
| | another physical attribute defined by DNA |
| | identifying dye Hoechst 33342. |
| Circularity_Head H Defined | Physical measure of the cell based on light |
| | scattering and distribution. |
| Count, Sperm w Tail (BF Defined) | Number of cells physically intact and with |
| | specific attributes as defined by bright field |
| | microscopy. |
| Count, Length, All & Sperm w Tail (BF | Number of cells physically intact and with |
| Defined) | specific attributes as defined by bright field |
| | microscopy. |
| Intensity_AGG, Whole_2-AGG, Mean, | A concentration of aggresomes combined |
| Intensity (all) +++ & Sperm w Tail (BF | with physical measure of the cell based on |
| Defined) | light scattering and distribution and |
| | including those cells that are physically |
| | intact as defined by bright field microscopy. |
| Aspect Ratio Intensity_M06_6-SSC | Physical measure of the cell based on light |
| | scattering and distribution in comparison to |
| | another physical attribute and a comparison |
| | of two of these aspects as a ratio. |
| Angle_M06 | Physical measure of the cell based on light |
| | scattering and distribution. |
| Gradient RMS_Head H Defined_7-H33342 | Physical measure of the cell based on light |
| | scattering and distribution in comparison to |
| | another physical attribute defined by DNA |
| | identifying dye Hoechst 33342. |
| Valley X_M06_6-SSC | Distance between two light distributions of |
| | a cell or group of cells. |
| Midpiece Length v3.0, Mean, Mid-length & | A physical measurement of one piece of a |
| Sperm w Tail (BF Defined) | sperm cell in a visually complete sperm cell. |
| Aspect Ratio_M06 | Physical measure of the cell based on light |
| | scattering and distribution. |
| Compactness_Head H Defined_7-H33342 | Physical measure of the cell based on light |
| | scattering and distribution in comparison to |
| | another physical attribute defined by DNA |
| | identifying dye Hoechst 33342. |
| Internalization_M06_6-SSC | Physical measure of the cell based on light |
| | scattering and distribution. |
| Symmetry 4_Head H Defined_7-H33342 | Physical measure of the cell based on light |
| | scattering and distribution in comparison to |
| | another physical attribute defined by DNA |
| | identifying dye Hoechst 33342. |
| Shape Ratio_M06 | Physical measure of the cell based on light |
| | scattering and distribution. |
| Shape Ratio_Head H Defined | Physical measure of the cell based on light |
| | scattering and distribution. |
| Mean Pixel_M06_6-SSC | Physical measure of the cell based on light |
| | scattering and distribution and pixilation of |
| | said measurement or image. |
| Aspect Ratio_Head H Defined | Physical measure of the cell based on light |
| | scattering and distribution. |
| Bright Detail Intensity R7_M06_6-SSC | Physical measure of the cell based on light |
| | scattering and distribution. |
| Count, Long & Sperm w Tail (BF Defined) | Number of cells that are long and are |
| | physically intact as determined by bright |
| | field microscopy. |
| Elongatedness_Head H Defined | Physical measure of the cell based on light |
| | scattering and distribution. |
| Midpiece Length v3.0, Mean, Length, | Number of cells physically intact and with |
| Exclude 0 & Sperm w Tail (BF Defined) | specific attributes as defined by bright field |
| | microscopy. |
| Bright Detail Intensity Low AGG | Physical measure of the cell based on light |
| | scattering and distribution and presence of |
| | aggresomes in a given concentration. |
| Modulation_M06_6-SSC | Physical measure of the cell based on light |
| | scattering and distribution. |
| Count, Short & Sperm w Tail (BF Defined) | Number of cells physically intact and with |
| | specific attributes as defined by bright field |
| | microscopy. |
| Midpiece Length v3.0, Mean, Long & Sperm | Number of cells physically intact and with |
| w Tail (BF Defined) | specific attributes as defined by bright field |
| | microscopy. |
| Length_Head H Defined, Mean, H Defined | Length of a sperm head or physical measure |
| Length | of the cell based on light scattering and |
| | distribution. |
| Gradient RMS_M01_1-BF | Physical measure of the cell based on light |
| | scattering and distribution and defined by |
| | bright field microscopy. |
| Spot Intensity Max_M06_6-SSC | Physical measure of the cell based on light |
| | scattering and distribution. |
| Saturation Count_M06_6-SSC | Physical measure of the cell based on light |
| | scattering and distribution. |
| Area_Head H Defined | Area of a sperm head defined by physical |
| | measure of the cell based on light scattering |
| | and distribution. |
| Lobe Count_Head H Defined_7-H33342 | Physical measure of the cell based on light |
| | scattering and distribution in comparison to |
| | another physical attribute defined by DNA |
| | identifying dye Hoechst 33342. |
| Centroid Y_M06 | Physical measure of the cell based on light |
| | scattering and distribution. |
| Width_M06 | Width of cell based on physical measure of |
| | the cell based on light scattering and |
| | distribution. |
| Raw Mean Pixel_M06_6-SSC | Physical measure of the cell based on light |
| | scattering and distribution and pixilation. |
| Major Axis_M06 | Physical measure of the cell based on light |
| | scattering and distribution. |
| Height_Head H Defined | Height of a component of the cell based on |
| | physical measure of the cell based on light |
| | scattering and distribution. |
| Centroid Y Intensity_M06_6-SSC | Physical measure of the cell based on light |
| | scattering and distribution. |
| Centroid X_M06 | Physical measure of the cell based on light |
| | scattering and distribution. |
| Bright Detail Intensity High AGG | Physical measure of the cell based on light |
| | scattering and distribution and presence of |
| | aggresomes. |
| Centroid X Intensity_M06_6-SSC | Physical measure of the cell based on light |
| | scattering and distribution. |
| Saturation Percent_M06_6-SSC | Physical measure of the cell based on light |
| | scattering and distribution. |
| Minor Axis_Head H Defined | Physical measure of the cell based on light |
| | scattering and distribution. |
| Bright Detail Intensity R3_M06_6-SSC | Physical measure of the cell based on light |
| | scattering and distribution. |
| H Entropy Mean_M06_6-SSC_5 | Physical measure of the cell based on light |
| | scattering and distribution. |
| Modulation_Head H Defined_7-H33342 | Physical measure of the cell based on light |
| | scattering and distribution in comparison to |
| | another physical attribute defined by DNA |
| | identifying dye Hoechst 33342. |
| Major Axis_Head H Defined | Physical measure of the cell based on light |
| | scattering and distribution. |
| H Variance Mean_M06_6-SSC_5 | Physical measure of the cell based on light |
| | scattering and distribution. |
| Width_Head H Defined | Width of cell portion; physical measure of |
| | the cell based on light scattering and |
| | distribution. |
| Intensity_M06_6-SSC, Mean, | Number of cells physically intact and with |
| Anteriorly/Posteriorly Aligned & Single | specific attributes as defined by presence of |
| Sperm Hoechst Defined & Focus | a specific stain. |
| Bright Detail Intensity Whole AGG | Physical measure of the cell based on light |
| | scattering and distribution and presence of |
| | aggresomes. |
| Lobe Count_M06_6-SSC | Physical measure of the cell based on light |
| | scattering and distribution. |
| Gradient Max_Head H Defined_7-H33342 | Physical measure of the cell based on light |
| | scattering and distribution in comparison to |
| | another physical attribute defined by DNA |
| | identifying dye Hoechst 33342. |
| Length_Head H Defined, Mean, Sperm w | Physical measure of the head parameters of |
| Tail (BF Defined) | the sperm cell that has an intact tail and is |
| | based on light scattering and distribution |
| | based on intact cell as defined by bright |
| | field microscopy. |
| Spot Distance Min_M06 | Physical measure of the cell or cell signal |
| | based on light scattering and distribution. |
| Length_M06 | Physical measure of the cell based on light |
| | scattering and distribution. |
| H Contrast Mean_M06_6-SSC_5 | Physical measure of the cell based on light |
| | scattering and distribution. |
| Elongatedness_M06 | Physical measure of the cell based on light |
| | scattering and distribution. |
| H Homogeneity Mean_M06_6-SSC_5 | Physical measure of the cell based on light |
| | scattering and distribution. |
| Thickness Max_M06 | Physical measure of the cell based on light |
| | scattering and distribution. |
| Minor Axis_M06 | Physical measure of the cell based on light |
| | scattering and distribution. |
| Area_M01 | Physical measure of the cell or cell signal |
| | based on light scattering and distribution. |
| Height_M06 | Physical measure of the cell based on light |
| | scattering and distribution. |
| Perimeter_M06 | Physical measure of the cell based on light |
| | scattering and distribution. |
| Area_M06 | Physical measure of the cell based on light |
| | scattering and distribution. |
| Diameter_M06 | Physical measure of the cell based on light |
| | scattering and distribution. |
| Acridine orange staining | Use of a DNA stain, acridine orange, to |
| | identify discrepancies in the DNA integrity |
| | of the cell. |
| High intensity DNA intact peak | Measure of DNA stain signals and the |
| | intensity of the signal. |
| Distance between left and middle peaks of | Physical measures of the distance between |
| healthy fluorescent population | populations of cells identified on a flow |
| | cytometer. |
| Coefficient of variance between populations | Statistical analysis of healthy sperm |
| of healthy sperm | populations. |
| Hoechst staining, Hoechst stained cell | Physical measure of the cell based on light |
| orientation, Hoechst fluorescent intensity | scattering and distribution defined by DNA |
| | identifying dye Hoechst 33342. |
| Standard deviation of peak width, height, | Statistical analysis of healthy sperm |
| difference between peak intensities, | populations. |
| Fluorescence peak area | Area of a data distribution as defined by |
| | fluorescence. |
| Difference between peak intensities | Difference between two or more data |
| | visualizations. |
| Total peak area of intact DNA fluorescence | Area under the curve for a particular type of |
| | cellular fluorescence data distribution. |
| Total peak area of functional or unfunctional | Cells deemed to be able to perform the |
| cell(s) | required function or not, and difference |
| | between these concentrations within a |
| | population or representative peak areas. |

The general health and even nutritional balance including but not limited to antioxidant deficiency, mineral deficiency, body condition score of the animal(s) involved in the reproductive process might be included as inputs, data, attributes such as but not limited to a mineral balance within the animal, trace mineral balance, and perhaps even nutritional balance or even health of the animal at the time of gamete collection, insemination, gestation, embryo transplantation, embryo implantation, or the like.

The various factors as listed and described herein may be weighted, may be assigned more or less importance, may be ranked, may be ordered, may be classified, or the like in the predictive equations. Techniques used to analyze data may include but is not limited to multiple levels or even layers of analysis, perhaps depending on the type of input or data, the type of animal to be utilized, the type of outcome desired, the type of assisted reproductive technology to be utilized, the desired outcome with respect to number of offspring, or the like. Parsing of data may be different between ejaculates, between or within groups of males, females, farms, data sets, between or within some type of circumstance or farm, or the like. Analysis may include, but is not limited to, statistical analysis; correlations; linear regressions; logistic regressions; A/B testing; random forest analysis or modeling; elasticity analysis; machine learning; natural language processing; neural networks; deep neural networks; all methods contained in neural networks such as convolutions and recurrence; similar types of statistical analysis and data analytics; or the like to create predictive outcomes. The predictive inference may utilize of a variety of statistical and even functional analyses. Data may be classified or even regularized using such models as Tikhonov regularization or Support Vector Machines, or the like.

Embodiments of the present invention may provide a variety of modeling techniques which may use a variety of models and even statistical analysis such that data might be fully and appropriately mined to generate decisions. Models and analysis may include, but is not limited to: time series algorithms such as exponential smoothing; regression algorithms such as linear regression; exponential regression; geometric regression; logarithmic regression; multiple linear regression; association algorithms such as Apriori; decision tree algorithms such as C 4.5 and CNR Tree; outlier detection algorithms such as inter quartile range and nearest neighbor outlier; neural network algorithms such as NNet Neural Network and MONMLP Neural Network; or the like. Additional models may include but are not limited to ensemble models; Fourier harmonics data analysis; factor analysis such as Maximum likelihood algorithm; probabilistic classifiers such as Bayes' theorem; uplift modeling; or the like. Any of these may be utilized singularly or in any combination. Prediction from each model may be considered individually or may be utilized to create a singular prediction. Modeling techniques may also include multilayer perceptron, gradient boost decision (e.g., XGBoost; Malik, Harode and Kunwar) type decision trees, or the like. Data utilized in modeling may be parametric or even non-parametric models and such models may be utilized within the same dataset perhaps to provide a range of alternative predictions. The models may use a combination of statistical and mathematical models and such models may be utilized at different intervals depending on the desired data outcome and decision-making assistance required.

Predictive equations or models may include imputation of data that may not be available for a given animal perhaps using data surrounding it or the data that is most similar. Such data may be imputed based on data from similar animals, conditions, within the same cohort, or the like. This type of analysis may include machine learning, causal analysis, predictive modeling, explanation of cause-and-effect relationships, predictions of various types of anticipated breeding results; or the like

In some embodiments, data may include limiting parameters such as the number of ejaculates evaluated, the number of breedings, the age of the animal, the type of artificial reproductive technology utilized, the inputs from the environment (e.g. the inseminator); or the like.

In other embodiments, data used to train a mathematical or even statistical predictive model may be selected from the same farm or farms or may be data from another similar or even related situation. The number of parameters included in a predictive equation can vary perhaps based on the species of animal, the output desired, the threshold for prediction required, the type of data available, or the like.

An output of the most preferable actions may be automatically generated when the predicted values may be generated which may provide a fully automated decision-making system.

An output may include analytics or even interpretation of data which may be used to guide or direct actions. Such actions or guidance may include but is not limited to: culling the male animal; use of specialized assisted reproductive technologies; decisions to breed to specific females; castration of the male animal; uses other than breeding; decisions to use or discard a specific ejaculate; decisions to use an ejaculate within a specific timeline; guidance on the type of female to be used in concert with the specific ejaculate or for a specific mating; guidance on the processing of the specific ejaculate such as dose concentration, cooling, freezing, freezing via specific methods; guidance on the types of media to be utilized with the specific ejaculate; or the like. Decisions made from data may be utilized to make alternative mating decisions such as adoption, surrogacy, use of donor gametes, limited or directed use of an ejaculate, use of in vitro fertilization, ICSI, use of donor sperm or oocytes, oocytes or implantation of an embryo, or the like. Data may provide: a method of deciding which male animals should be removed perhaps using characteristics of sperm quality and/or fertility; a method of creating matings perhaps based on predicted results of such matings or predicted mating success; a method of predicting the result of breeding specific male and female animals perhaps based on a single or combined sperm quality data, oocyte quality data, embryo quality data; a method of making intelligent, informed decisions on use of specific assisted reproductive technologies; or the like.

### EXAMPLE 1

Predicting Conception Rate. In an experiment, four ejaculates were collected on specific dates then analyzed for motility, morphology, acrosome quality, DNA quality, membrane quality, membrane fluidity, mitochondrial potential, antioxidant capacity, mean fluorescence of SYBR (SYBR green), Alexafluor 647, pH and osmolality of the extended sample and extender type. These ejaculates were used to breed a minimum of 25 sows. The sperm data was analyzed for fertility predictions using a random forest model trained on a subset of data then tested against another set of data from these breedings. Table 2 provides these experimental results which include the prediction and the accuracy of the prediction as well as a presumptive decision about future use of the boar within the production system (e.g., fertility use and culling decision).

TABLE 2

| | Fertility prediction | | | | |
|---|---|---|---|---|---|
| | (Conception Rate | | Genetic | Fertility | Culling |
| Animal ID | Accuracy r²) | Score | merit | use | decision |
| Boar 1 | 0.917166599 | 0.779 | 0.5 | X | |
| Boar 2 | 0.897253195 | 0.730 | 0.4 | | X |
| Boar 3 | 0.741341981 | 0.602 | 0.8 | X | |
| Boar 4 | 0.562800113 | 0.367 | 0.8 | | X |
| Boar 5 | 0.624677979 | 0.424 | 0.8 | X | |
| Boar 6 | 0.731452037 | 0.534 | 0.7 | X | |

For prediction of conception rates, the following parameters could be weighted as influential within a model utilized in any combination or permutation:
Percentage of sperm cells that are membrane intact;
Percentage of sperm cells that are acrosome intact;
Percentage of sperm cells that are acrosome and membrane intact;
Percentage of sperm cells that have compromised or broken (single or double stranded breaks) DNA;
Number of intact sperm cells that are mid-length or midpiece length and have tails;
Minor axis intensity of measured cells;
Ratio of oxidized to basal induced (TAR value);
Percentage of cells that are negative for merocyanin staining; and perhaps even
Percentage of cells stained with Hoechst 33342 with symmetry 3.

As can be understood from Table 1, the use of analysis of a variety of parameters of an ejaculate can be used to predict the conception rate when using a particular ejaculate. The data can then be utilized to determine if a particular boar should be utilized for future breeding or to be culled from the herd.

### EXAMPLE 2

Predicting Total Born. In a model, a prediction for the number of total piglets born to a sow based on characteristics of an ejaculate and sperm cells within were analyzed to provide the values in Table 3. Table 3 shows the prediction of total number of piglets born in a litter based on semen qualities.

TABLE 3

| Predicted total born | R² | Angle | Score |
|---|---|---|---|
| 12 piglets | 0.923813482 | 39.59118 | 0.824689 |

For prediction of total born, the following parameters could be weighted as influential within a model utilized in any combination or permutation:
Percentage of sperm cells that have proximal droplets;
Percentage of sperm cells have distal droplets;
Percentage of sperm cells that have high mitochondrial activity;
Percentage of cells that have depolarized mitochondria;
Percentage of cells with distal midpiece reflex;
Percentage of cells that are motile;
Percentage of cells that are morphological normal;
Percentage of mercyanin negative and are in transition (unstable shifting);
Percentage of sperm cells that have compromised or broken (single or double stranded breaks) DNA;
Median beat frequency of motile cells;
Percentage of cells swimming slowly;
Percentage of abnormal cells;
Total concentration of cells in a dose;
Percentage of cells that have intact DNA (no breaks);
Percentage of cells that are transitioning between stable to unstable membrane;
Percentage of cells that are membrane permeable and have intact acrosomes; and perhaps even
Medial straightline velocity of motile cells.

### EXAMPLE 3

Predicting number born alive. In a model, a prediction for the number of piglets born alive born to a sow based on characteristics of an ejaculate and sperm cells within were analyzed to provide the values in Table 4. Table 4 shows the prediction of total number of piglets born live in a litter based on semen qualities.

TABLE 4

| Number born alive | R² | Angle | Score |
|---|---|---|---|
| 11 piglets | 0.924011882 | 41.40863 | 0.855718 |

For prediction of number born alive, the following parameters could be weighted as influential within a model utilized, perhaps to determine the predictions within a random forest, in any combination or permutation:
Total percent of motile sperm;
Percent of sperm having distal droplets;
Percent of sperm swimming progressively;
Percent of Motile sperm swimming slowly;
Percent morphologically normal;
Motile median straightness;
Concentration;
Proximal droplet count;
Distal midpiece reflex count;
Motile median beat frequency of motile sperm;
Motile mean straightness;
Motile mean beat frequency;
Motile median curvilinear velocity; and perhaps even
Abnormal head count.

In a study, analysis of both US swine and bovine herds show that variation in pregnancy rates may be more attributable to male-factor subfertility and infertility than the dam. To date, a limited degree of correlations may be observed between conventional semen analysis parameters and actual fertility after standard quality cutoffs may be met. Thus, a clear ability to predict male-factor fertility may be lacking. In vitro capacitation-induced changes to sperm zinc signature could be indicative of male-factor sub- and infertility. A fertility trial included 210 boar ejaculates from 66 boars inseminated to 2508 sows in a single, fixed-time artificial insemination setting, with pregnancy results ranging from about 56.4% to 96.8%. Each ejaculate underwent in vitro capacitation with 10,000 spermatozoa imaged at 1, 1, and 4 hours utilizing high throughput, image-based flow cytometry. Over 6550 bioimages values were calculated for each of the time points analyzed. Mutual information analysis found 27 sperm bioimage features with scores greater than 0.1 mutually informative to the pregnancy rate. Linear regression analysis was performed on each of these features and tested with a nested model. ANOVA of the linear regression model identified four features significant with high fertile males within a nested model and eight features for the full model. The four features for the nested, high pregnancy included: 0 hour, Centroid Y Intensity M07_7-H33342, Mode; 0 hour, Contrast MC_1-BR Median; 4 hour, H Correlation Std_M32_2-Zn_5, Mode; and 4 hour, Min Pixel_Valley(M36,7-H33342,3)_7-H33342,MAD. The eight features for the whole model included: 0 hour, Centroid Y Intensity M07_7-H33342, Mode; 0 hour, Contrast MC_01-BR Median; 0 hour, Contrast MC_1-BR Median; 0 hour, Elonness_Object(M07,7-H33342, Tight), Mode; 0 hour, Valley X_MC_6-55C,MAC; 4 hour, Area_Skeleton(Object(Mo2,2-ZN,Tight),2-Zn,Thin), Median; 4 hour, H Correlation Std_M22_2-Zn_5, Mode; 4 hour, H Entropy Std_MO':_': 1:1_11, Mode.

Next, the data was randomly split (4:1) into training and testing sets and classification trees were calculated to predict the pregnancy rates after being discretized into fertile (about 85% pregnancy rates) and sub/infertile classes (below 80% pregnancy). One tree was trained with 17 features related to morphology and computer-assisted sperm analysis (CASA) motility outputs (e.g., a traditional model), and a separate tree was trained with 170 features related to differences in subpopulation changes to the sperm zinc signature, acrosomal modifications, and plasma membrane integrity after in vitro semen capacitation after one and four hours, including motility and features implicated by mutual information analysis. The traditional model yielded respective training and testing accuracies of 100% and 53.8%, whereas the subpopulation/change model yielded respective training and testing accuracies of 100% and 77.9%. Here, it was identified that the ability for sperm to transition from a zinc signature 1 and 2 to a capacitated state signature 3 and 4 along with acrosomal modification and changes to a plasma membrane integrity may excel in predictive value of male factor fertility compared to traditional motility and morphology scores alone. Such findings may establish a new paradigm in the role of zinc ions in sperm function and pave the way for accurate sperm biomarker identification of male factor sub/infertility in future precision agriculture and medicine applications.

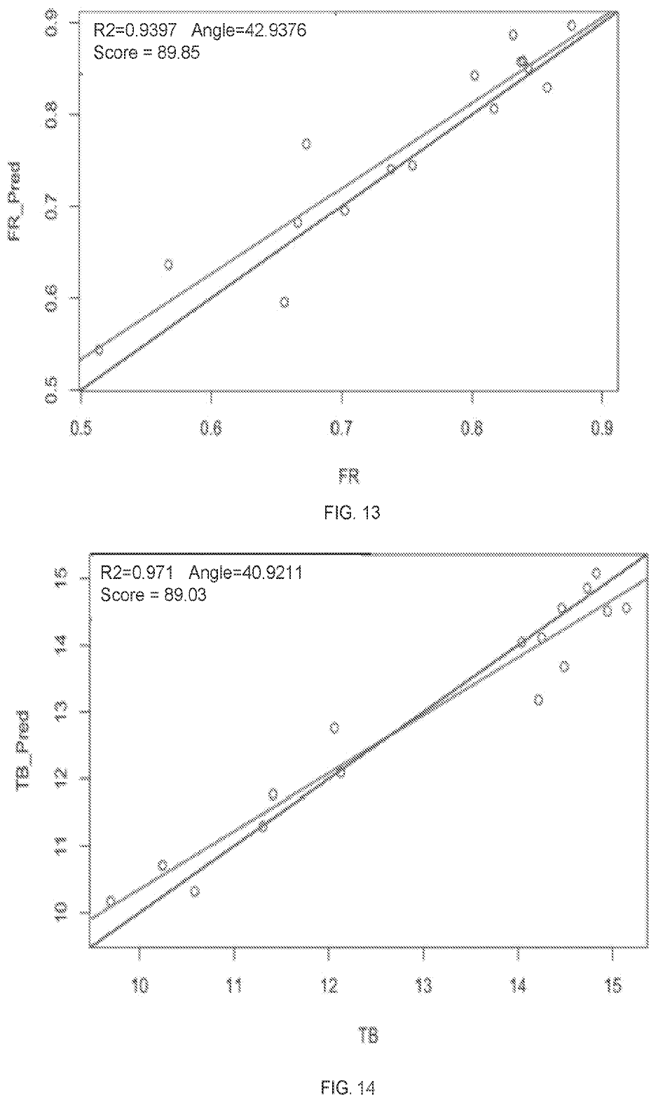

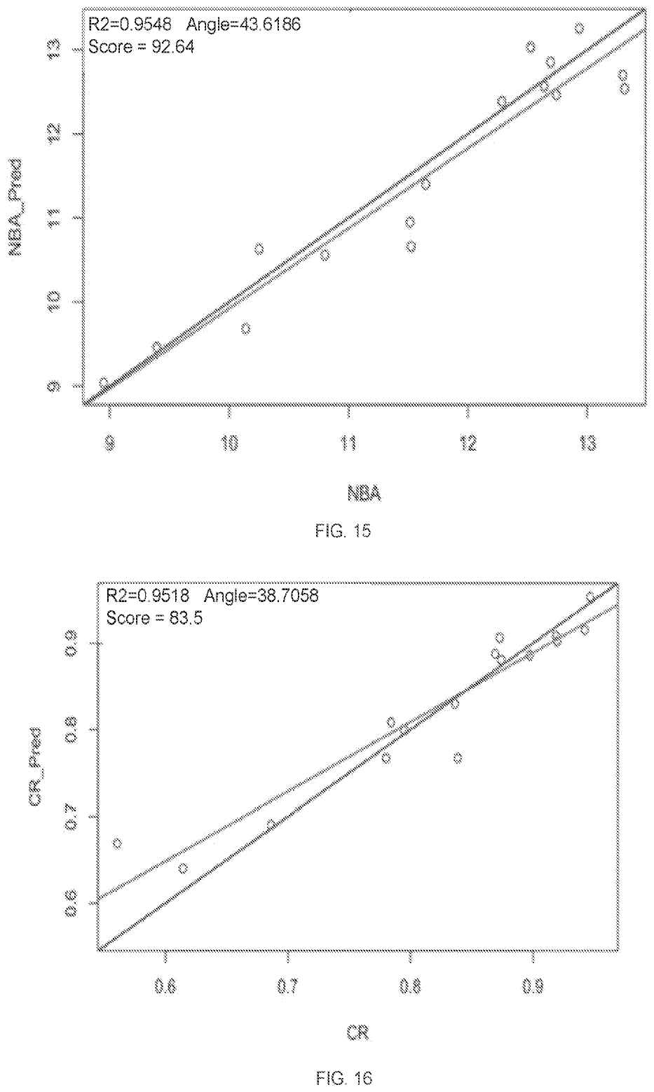

FIGS. 13-16 show the effect of using qualities, attributes, characteristics, inputs and even physical characteristics to predict a farrowing rate, total born, number born alive, and conception rates. A perfect correlation-line is drawn along with the predictive ability of the specific combination (r² value) for a method of prediction.

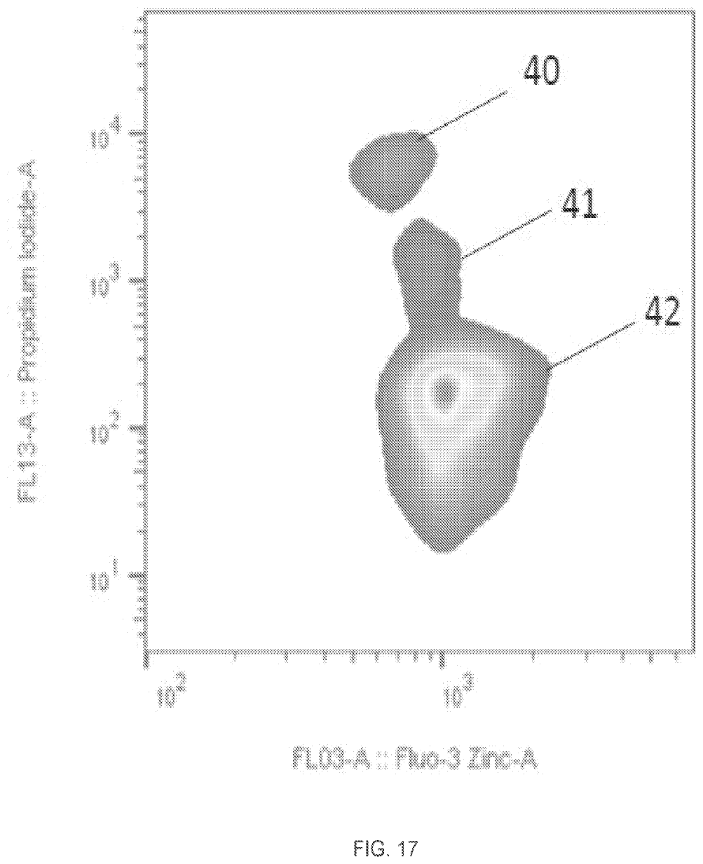

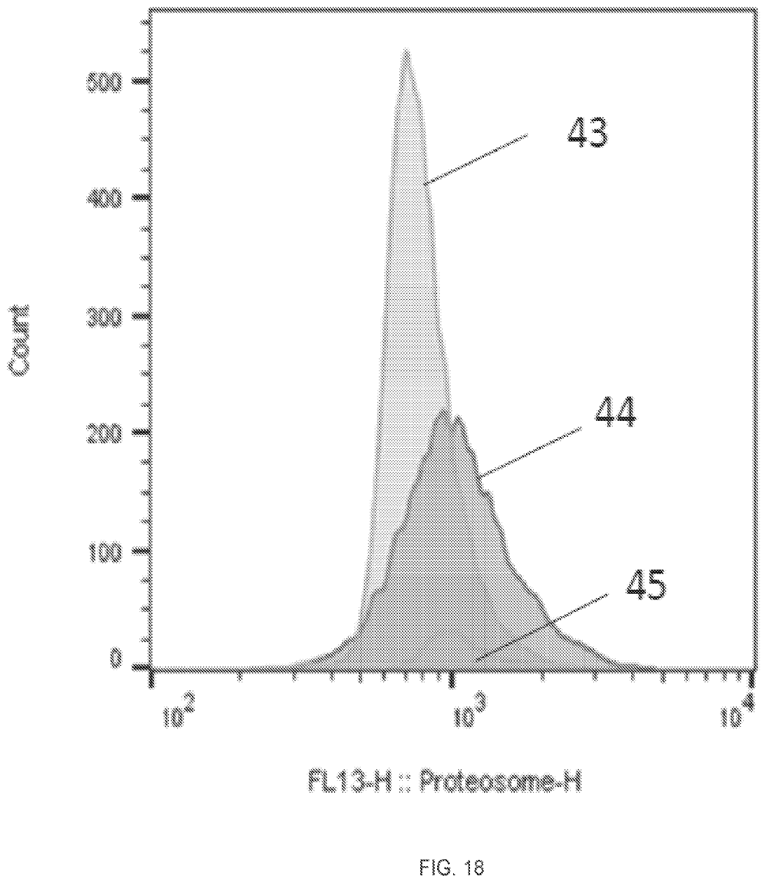

FIGS. 17-20 shows a non-limiting example of results of different assays that may be utilized to assess sperm quality and demonstrates different responses of individual sperm cells to the assays. FIG. 17 shows the results of a flow cytometric assay of zinc concentration within the cell where population (42) has high zinc (median fluorescence 10000), population (41) is in transition and has a median fluorescence of 8000 and population (40) has lost zinc and has a median fluorescence of 7000. FIG. 18 shows the different populations of sperm cells with different levels of aggresomes (ubiquitin containing proteins). Peak (43) are live, healthy sperm cells with few aggresomes and low fluorescence (median fluorescence 5000) while peak (45) is dead sperm having more fluorescence (median fluorescence >10,000). Peak (44) is a positive control demonstrating the assay is working as expected.

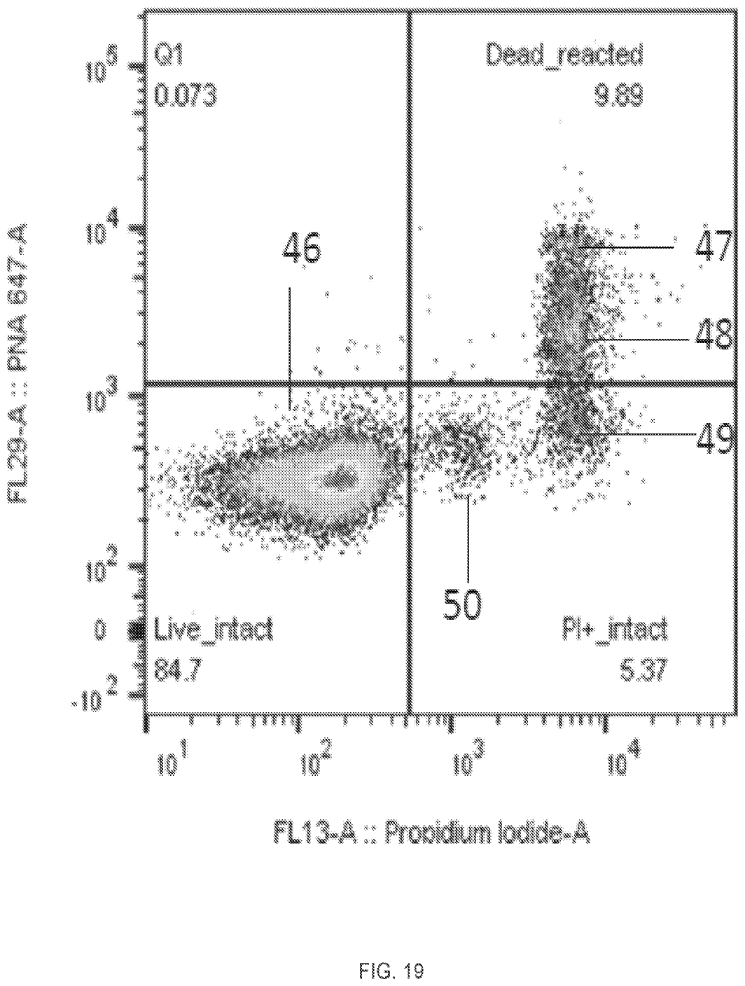

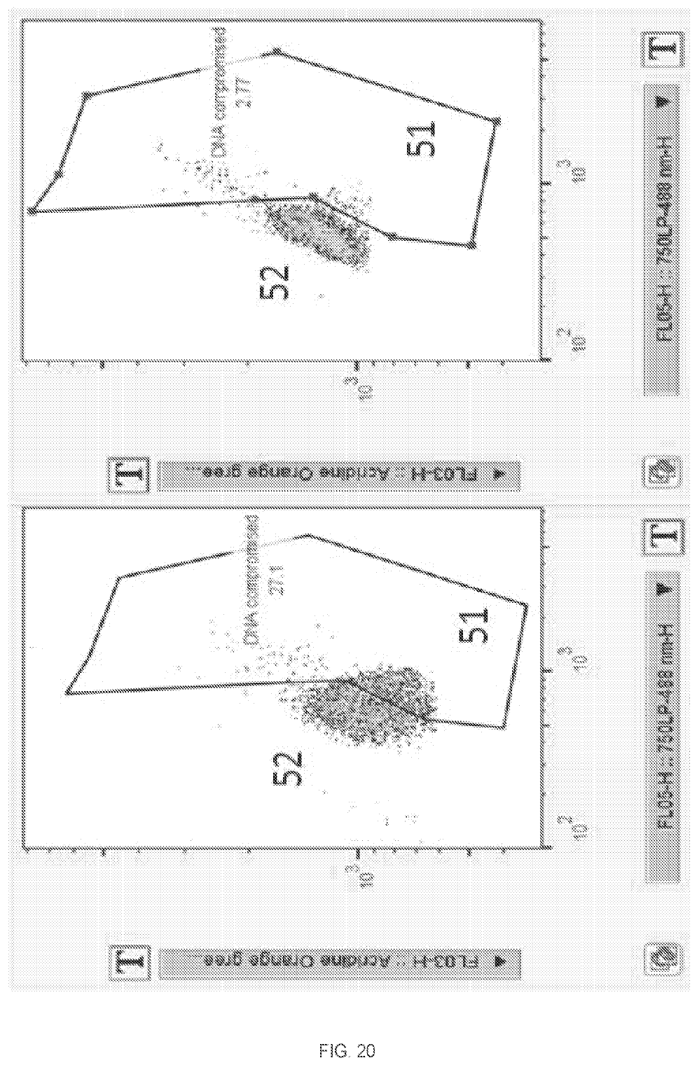

FIG. 19 is a flow cytometry dot plot showing the fluorescence of individual sperm cells (individual dots =1 measured cell) when assaying for both intact acrosomes (Y-Axis) and membrane quality (X-Axis). Population (46) is low in fluorescence of both acrosome and membrane permeable stains indicating the cells have intact acrosomes and membranes. The top-right quadrant has two populations that have reacted acrosomes although population (48) has fewer than population (47). Populations (49) and (50) are in transition. FIG. 20 is a flow cytometry dot plot demonstrating the identification of DNA fragmentation. DNA fragmentation (51) has greater fluorescence than a normal population (non-fragmented (52)).

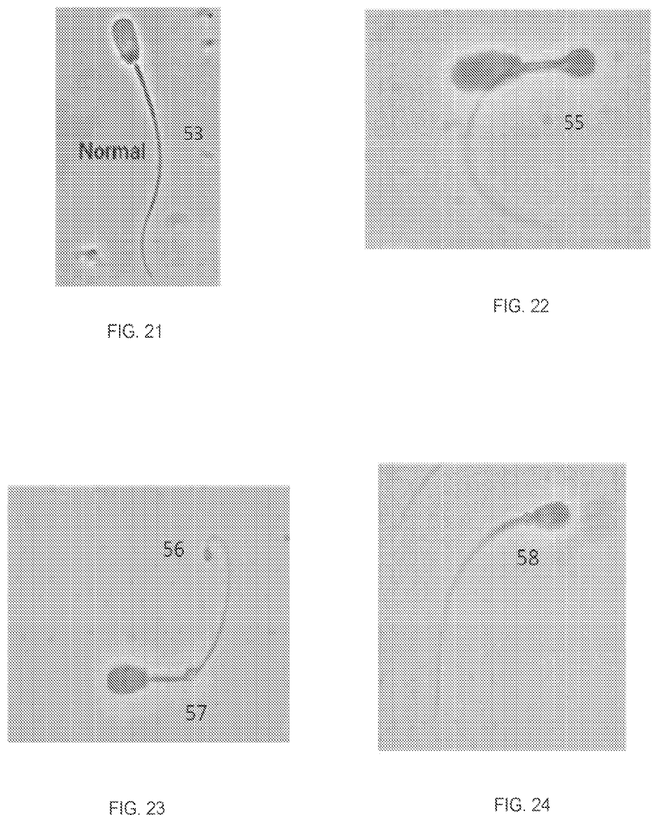

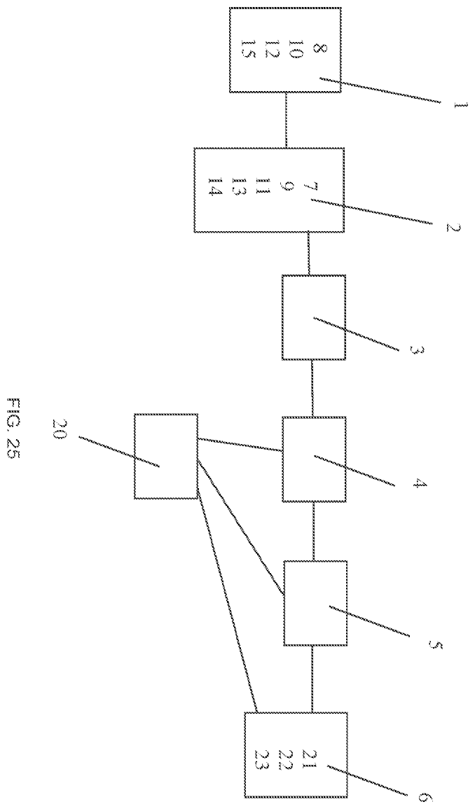

FIGS. 21-24 show pictures of cells with different morphological characteristics. FIG. 21 shows a morphologically normal sperm (53). FIGS. 22-24 demonstrates abnormalities such as a distal midpiece reflex (55), a droplet (57), a coiled tail (56) and a proximal droplet (58).

As can be determined using the aforementioned examples, a variety of inputs may be utilized perhaps in a weighted fashion to accurately predict different variables. It should be noted that the non-limiting examples herein contain ONLY the top about 10 or about 20 variables utilized while a total of about 100 or more may be initially input into the model. While their weight may be less than the top about 10 or about 20 variables, they may be of utmost importance in creating an accurate prediction. Moreover, inputs may vary between species, subspecies, between different livestock types and uses, or the like. In addition, for predicting other variables such as success in an IVF system for example, or perhaps even for different cell types and predictions, a vastly different set of inputs may be required.

While the invention has been described in connection with some preferred embodiments, it is not intended to limit the scope of the invention to the particular form set forth, but on the contrary, it is intended to cover such alternatives, modifications, and equivalents as may be included within the spirit and scope of the invention as defined by the statements of invention.

Examples of alternative claims may include:
1. A method of efficient fertility prediction comprising the steps of:
   evaluating semen qualities from an ejaculate of a male animal; and
   predicting fertility-related parameters of said ejaculate of said male animal.
2. A method of efficient fertility prediction comprising the steps of:
   evaluating semen qualities from an ejaculate of a male animal;
   establishing in a computational device prediction models automated computational transformation algorithm;
   automatically applying said prediction models automated computational transformation algorithm to said semen qualities to automatically create prediction model transformed data of said semen qualities;
   generating prediction models completed prediction output based on said prediction model transformed data of said semen qualities; and
   predicting fertility-related parameters of said ejaculate of said male animal based on said prediction models completed prediction output.
3. The method as described in clause 2 or any other clause wherein said prediction models automated computational transformation algorithm comprises models chosen from statistical models, mathematical models, machine learning models, regression analyses, and any combination thereof.
4. The method as described in clause 2 or any other clause and further comprising the step of using said fertility-related parameters in making a decision based on said fertility-related parameters.
5. The method as described in clause 4 or any other clause wherein said decision is chosen from a breeding decision, a culling decision, and a type of assisted reproductive technology used with said ejaculate of said male animal.
6. The method as described in clause 2 or any other clause wherein said semen qualities are chosen from semen state, animal information of said male animal, gross sperm information, morphological aspects, cellular motion, cellular function, regulation of intracellular information, reduction-oxidation balance, population of cells, sperm developmental conditions, personnel skills, and any combination thereof.
7. The method as described in clause 6 or any other clause wherein said semen state is chosen from fresh sperm, frozen sperm, cooled sperm, vitrified sperm, sperm in gel state, thawed sperm, extended sperm, warmed sperm, freeze dried sperm, dehydrated sperm, rehydrated sperm, in vitro sperm, epidydimal sperm, multiple sperm cells, and singular sperm cells.
8. The method as described in clause 6 or any other clause wherein said animal information comprises information chosen from identification of a particular animal, testosterone level, weight, nutritional status, genetic line, body condition score, breeding soundness exam, transportation, stress of said male animal, hemoglobin, fibronectin, inflammation markers, method of collection of sperm, electrical conductivity of sperm, presence of metal ions, concentration of sperm, hormone levels, temperature, and any combination thereof.
9. The method as described in clause 6 or any other clause wherein said gross sperm information comprises information chosen from seminal plasma information, electrical conductivity of fluid, storage period, storage temperature, refractometry, thermoresistance, type of solutions, solution characteristics, contaminants, contaminant concentration, organism contaminating, and any combination thereof.
10. The method as described in clause 6 or any other clause wherein said morphological aspects comprises information chosen from physical characteristics of cells, percentage normal cells, percentage abnormal cells, shape descriptions, volume, aspect ratios, ratios of physical sperm attributes to one another, total mass, concentration of sperm relative to seminal plasma, length, width, area, thickness, midpiece defects, abnormal heads, distal midpiece reflex, and any combination thereof.
11. The method as described in clause 6 or any other clause wherein said cellular motion comprises information chosen from total motility, progressive motility, velocity descriptors, rate of motility, velocity of motility, percentage of cells in each velocity category, kinematic parameters, mean, median and mode of kinematic parameters, agglutination, and any combination thereof.
12. The method as described in clause 6 or any other clause wherein said cellular function comprises information chosen from acrosome quality, membrane quality, membrane fluidity, mitochondrial quality and depolarization, presence of aggresomes, ubiquitin, ubiquitinated proteins, zinc, zinc concentration, apoptotic cells, DNA quality, reciprocal translocations, single nucleotide polymorphism (SNP), seminal plasma proteins, data distribution differences, mitochondrial depolarization, and any combination thereof.
13. The method as described in clause 6 or any other clause wherein said regulation of intracellular information comprises information chosen from cAMP, MTOR-pathways, mineral composition, metal, zinc, calcium, cortisol, serotonin, hippocampal glucocorticoid, and any combination thereof.
14. The method as described in clause 6 or any other clause wherein said reduction-oxidation balance comprises information chosen from total antioxidant capacity of cells, total antioxidant capacity of extender, total antioxidant capacity of seminal plasma, total antioxidant capacity intracellular, total antioxidant capacity extracellular, superoxide dismutase concentration, endogenous and exogenous antioxidants, presence of oxidants intracellular, presence of oxidants extracellular, presence of antioxidants intracellular, presence of antioxidants extracellular, membrane reduction-oxidation balance, oxidative damage, reactive oxygen species, reactive sulfur species, reactive nitrogen species, and any combination thereof.
15. The method as described in clause 6 or any other clause wherein said population of cells comprises information chosen from intensity of fluorescence of membrane, intensity of fluorescence of DNA, intensity of fluorescence of acrosomes, intensity of fluorescence of membrane fluidity, delta between fluorescent populations, median of fluorescence intensity for a specific population, mode of fluorescence intensity for a specific population, and any combination thereof.
16. The method as described in clause 6 or any other clause wherein said sperm developmental conditions comprises information chosen from temperature during spermatogenesis, temperature during spermiogenesis, humidity during spermatogenesis, humidity during spermiogenesis, temperature during collection, temperature during insemination, temperature of solutions, barometric conditions, barometric pressure, temperature of testicles, and any combination thereof.
17. The method as described in clause 6 or any other clause wherein said personnel skills comprises information chosen from a technician's skills when thawing said semen, a technician's skills when handling said semen, a technician's skills when warming said semen, a technician's skills when inseminating said semen, and any combination thereof.
18. The method as described in clause 2 or any other clause wherein said fertility-related parameters comprises a parameter chosen from conception rate, parturition rate, total number of animals born alive, and total number of animals born.
19. The method as described in clause 18 or any other clause wherein said fertility-related parameters comprises a parameter chosen from calving rate, foaling rate, farrowing rate, kidding rate, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, and any combination thereof.
20. The method as described in clause 2 or 3 or any other clause wherein said step of predicting said fertility-related parameters of said ejaculate of said male animal based on said prediction models completed prediction output comprises a step of generating a numeric indication of said fertility-related parameters.
21. The method as described in clause 20 or any other clause wherein said step of generating said numeric indication of said fertility-related parameters comprises a step of utilizing a r² value from a regression analysis to predict said fertility-related parameters.
22. The method as described in clause 20 or 21 or any other clause wherein said step of generating said numeric indication of said fertility-related parameters comprises a step of evaluating an angle of a line in comparison to a perfect 45-degree angle from a regression analysis and utilizing said comparison to predict said fertility-related parameters.
23. The method as described in clause 22 or any other clause and further comprising the step of combining said r² value and said angle of said line in said comparison to a perfect 45-degree angle to create a combined score and using said combined score in said step of utilizing said comparison to predict said fertility-related parameters.
24. The method as described in clause 20 or any other clause wherein said step of generating said numeric indication of said fertility-related parameters comprises a step of utilizing variances to predict said fertility-related parameters.
25. The method as described in clause 24 or any other clause wherein said variances are chosen from variances relative to another population, variances relative to populations within a sample, variances relative to other samples taken from a same animal, variances relative to samples taken from related animals, variances within a population, and any combination thereof.
26. The method as described in clause 20 or any other clause wherein said step of generating a numeric indication of said fertility-related parameters comprises using a mathematical property chosen from Rho correlation, delta of means, accuracy, precision, contingency table, and any combination thereof.
27. The method as described in clause 2 or any other clause wherein said animal is chosen from bovine, equine, ovine, porcine, caprine, avian, and human.
28. The method as described in clause 2 or any other clause wherein said prediction models automated computational transformation algorithm comprises a trained, automatically self-improving algorithm based on existing data.
29. The method as described in clause 2 or any other clause and further comprising a step of using one or more different semen qualities with said prediction models automated computational transformation algorithm.
30. The method as described in clause 2 or any other clause and further comprising the steps of:
   evaluating female characteristics from a female animal;
   automatically applying said prediction models automated computational transformation algorithm to said female characteristics to automatically create said prediction model transformed data of said semen qualities and said female characteristics; and
   generating said prediction models completed prediction output based on said prediction model transformed data of said semen qualities and said female characteristics.
31. A method of efficient fertility prediction comprising the steps of:
   evaluating female characteristics of a female animal;
   establishing in a computational device prediction models automated computational transformation algorithm;
   automatically applying said prediction models automated computational transformation algorithm to said female characteristics to automatically create prediction model transformed data;
   generating prediction models completed prediction output based on said prediction model transformed data of said female characteristics; and
   predicting fertility-related parameters of said female animal based on said prediction models completed prediction output.
32. The method as described in clause 31 or any other clause wherein said step of predicting fertility-related parameters of said female animal does not include any data from a male animal.
33. The method as described in clause 31 or any other clause wherein said prediction models automated computational transformation algorithm comprises models chosen from statistical models, mathematical models, machine learning models, regression analyses, and any combination thereof.
34. The method as described in clause 31 or any other clause and further comprising the step of using said fertility-related parameters in making a decision based on said fertility-related parameters.
35. The method as described in clause 34 or any other clause wherein said decision is chosen from a breeding decision, a culling decision, and a type of assisted reproductive technology used with said female animal
36. The method as described in clause 31 or any other clause wherein said female characteristics are chosen from oocyte type, animal information of said female animal, gross gamete information, morphological aspects, cellular function, regulation of intracellular information, reduction-oxidation balance, population of cells, oocyte developmental conditions, personnel skills, and any combination thereof.
37. The method as described in clause 36 or any other clause wherein said oocyte type is chosen from fresh oocytes, frozen oocytes, cooled oocytes, superovulated oocytes, flushed oocyte, vitrified oocyte, thawed oocyte, freeze dried oocytes, warmed oocyte, donor oocyte, in vivo oocyte, in vitro oocyte, in utero oocyte and any combination thereof.
38. The method as described in clause 36 or any other clause wherein said animal information comprises information chosen from weight, nutritional status, body condition score, parturition information, weaning information, breeding schedule, parity, body temperature, vulvar temperature, uterine temperature, transportation, stress of said female animal, synchronization protocol, hemoglobin, fibronectin, inflammation markers, method of collection of oocytes, hormone levels, uterine health, reproductive health, involution state of uterus, follicle health, follicle maturity, donor information, recipient information, temperature, and any combination thereof.
39. The method as described in clause 36 or any other clause wherein said gross gamete information comprises information chosen from follicular fluid, electrical conductivity of fluid, storage period, storage temperature, refractometry, thermoresistance, type of solutions, solution characteristics, oxygen saturation of solutions, contaminants, synchronization protocol, follicle health, follicle maturity, oocyte health, and any combination thereof.
40. The method as described in clause 36 or any other clause wherein said morphological aspects comprises information chosen from physical characteristics of oocyte cells from said female animal, shape descriptions of oocytes from said female animal, volume of oocytes from said female animal, ratios of cellular dimensions, distributions, compactness, total mass of oocytes from said female animal, cumulus cell to oocyte mass, number of oocytes/dish, spindle formation, chromosome location and position, mitochondrial distribution, organelle distribution, percentage of normal oocytes from said female animal, length of oocytes from said female animal, width of oocytes from said female animal, area of oocytes from said female animal, and any combination thereof.
41. The method as described in clause 36 or any other clause wherein said cellular function comprises information about oocytes from said female animal chosen from membrane quality, membrane fluidity, aggresomes, ubiquitin, ubiquitinated proteins, zinc concentration, apoptotic cells, spindle formation, DNA quality, reciprocal translocations, cellular division status, oviductal cell binding, single nucleotide polymorphism (SNP), organelle distribution, data distribution differences, mitochondrial depolarization, and any combination thereof.
42. The method as described in clause 36 or any other clause wherein said regulation of intracellular information comprises information chosen from cAMP, MTOR-pathways, mineral composition, metal, zinc, calcium, cortisol, serotonin, hippocampal glucocorticoid, presence of ubiquitin, presence of zinc, cell surface markers, and any combination thereof.
43. The method as described in clause 36 or any other clause wherein said reduction-oxidative comprises information chosen from total antioxidant capacity of cells, total antioxidant capacity of fluids, total antioxidant capacity intracellular, total antioxidant capacity extracellular, intracellular antioxidants, extracellular antioxidants, presence of oxidants intracellular, presence of oxidants extracellular, membrane reduction oxidative balance, presence of CD9, oxidative damage, reactive oxygen species, presence of reactive nitrogen species, presence of reactive sulfur species, and any combination thereof.
44. The method as described in clause 36 or any other clause wherein said population of cells comprises information about said oocytes from said female animal chosen from intensity of fluorescence of membrane, intensity of fluorescence of DNA, intensity of fluorescence of acrosomes, intensity of fluorescence of membrane fluidity, delta between fluorescent populations, average fluorescence intensity between a group of cells, average fluorescence intensity of individual cells, and any combination thereof.
45. The method as described in clause 36 or any other clause wherein said oocyte developmental conditions comprises information chosen from temperature during oogenesis, humidity during oogenesis, temperature during oocyte collection, temperature during oocyte culture, humidity during oocyte collection and culture, a change in temperature, the timeline between critical events within the process, temperature during insemination, temperature during culture, temperature of solutions, barometric conditions, and any combination thereof.
46. The method as described in clause 36 or any other clause wherein said personnel skills comprises information chosen from a technician's skills when thawing oocytes from said female animal, a technician's skills when handling oocytes from said female animal, a technician's skills when warming oocytes from said female animal, a technician's skills when implanting, a technician's skills when culturing, and any combination thereof.
47. The method as described in clause 31 or any other clause wherein said fertility-related parameters comprises a parameter chosen from conception rate, parturition rate, total number of animals born alive, and total number of animals born.
48. The method as described in clause 47 or any other clause wherein said fertility-related parameters comprises a parameter chosen from farrowing rate, kidding rate, oocyte quality, oocyte fertility potential, oocyte health, post-thaw oocyte health, calving rate, foaling rate, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, and any combination thereof.
49. The method as described in clause 31 or 33 or any other clause wherein said step of predicting fertility of said female animal based on said prediction models completed prediction output comprises a step of generating a numeric indication of said fertility-related parameters.
50. The method as described in clause 49 or any other clause wherein said step of generating said numeric indication of said fertility-related parameters comprises a step of utilizing a r² value from a regression analysis to predict said fertility-related parameters.
51. The method as described in clause 49 or 50 or any other clause wherein said step of generating said numeric indication of said fertility-related parameters comprises a step of evaluating an angle of a line in comparison to a perfect 45-degree angle from a regression analysis and utilizing said comparison to predict said fertility-related parameters.
52. The method as described in clause 51 or any other clause and further comprising the step of combining said r² value and said angle of said line in said comparison to a perfect 45-degree angle to create a combined score and using said combined score in said step of utilizing said comparison to predict said fertility-related parameters.
53. The method as described in clause 49 or any other clause wherein said step of generating said numeric indication of said fertility-related parameters comprises a step of utilizing variances to predict said fertility-related parameters.
54. The method as described in clause 53 or any other clause wherein said variances are chosen from variances relative to another population, variances relative to populations within a sample, variances relative to other samples taken from a same animal, variances relative to samples taken from related animals, variances within a population, and any combination thereof.
55. The method as described in clause 49 or any other clause wherein said step of generating a numeric indication of said fertility-related parameters comprises using a mathematical property chosen from Rho correlation, delta of means, accuracy, precision, contingency table, and any combination thereof.
56. The method as described in clause 31 or any other clause wherein said animal is chosen from bovine, equine, ovine, porcine, caprine, avian, and human.
57. The method as described in clause 31 or any other clause wherein said prediction models automated computational transformation algorithm comprises a trained, automatically self-improving algorithm based on existing data.
58. The method as described in clause 31 or any other clause and further comprising a step of using one or more different female characteristics with said prediction models automated computational transformation algorithm.
59. The method as described in clause 31 or any other clause and further comprising the steps of:
   evaluating semen qualities from a male animal;
   automatically applying said prediction models automated computational transformation algorithm to said semen qualities to automatically create said prediction model transformed data; and
   generating said prediction models completed prediction output based on said prediction model transformed data of said semen qualities and said female characteristics.
60. A method for efficient fertility prediction comprising the steps of:
   evaluating embryo characteristics of an embryo; and
   predicting an embryo success rate of said embryo.
61. A method for efficient fertility prediction comprising the steps of:
   evaluating embryo characteristics of an embryo;
   establishing in a computational device prediction models automated computational transformation algorithm;
   automatically applying said prediction models automated computational transformation algorithm to said embryo characteristics to automatically create prediction model transformed data;
   generating prediction models completed prediction output based on said prediction model transformed data of said embryo characteristics; and
   predicting an embryo success rate of said embryo based on said prediction models completed prediction output.
62. The method as described in clause 61 or any other clause wherein said prediction models automated computational transformation algorithm comprises models chosen from statistical models, mathematical models, machine learning models, regression analyses, and any combination thereof.
63. The method as described in clause 61 or any other clause and further comprising the step of using said embryo success rate in making a decision based on said embryo success rate.
64. The method as described in clause 63 or any other clause wherein said decision is chosen from a breeding decision, a culling decision, and a type of assisted reproductive technology used with said embryo.
65. The method as described in clause 61 or any other clause wherein said embryo characteristics are chosen from gamete type, animal information, gross gamete information, morphological aspects, cellular function, regulation of intracellular information, reduction-oxidation balance, population of cells, gamete developmental conditions, personnel skills, and any combination thereof.
66. The method as described in clause 65 or any other clause wherein said gamete type is chosen from fresh, frozen, cooled, flushed embryo, superovulated oocytes, flushed oocyte, and any combination thereof.
67. The method as described in clause 65 or any other clause wherein said animal information comprises information chosen from weight, nutritional status, body condition score, parturition information, breeding schedule, parity, transportation, stress of an animal, synchronization protocol, hemoglobin, fibronectin, inflammation markers, method of collection, hormone levels, donor information, recipient information, temperature, and any combination thereof.
68. The method as described in clause 65 or any other clause wherein said gross gamete information comprises information from gametes used to create said embryo chosen from follicular fluid, electrical conductivity of fluid, storage period, storage temperature, refractometry, thermoresistance, type of solutions, solution characteristics, contaminants, synchronization protocol, follicle health, follicle maturity, oocyte health, and any combination thereof.
69. The method as described in clause 65 or any other clause wherein said morphological aspects comprises information chosen from physical characteristics of said embryo, shape descriptions of said embryo, volume of said embryo, ratios of physical attributes to one another, total mass of said embryo, mitochondrial distribution, organelle distribution, percentage of normal cells, length of said embryo, width of said embryo, area of said embryo, and any combination thereof.
70. The method as described in clause 65 or any other clause wherein said cellular function comprises information about said embryo chosen from membrane quality, membrane fluidity, aggresomes, ubiquitin, ubiquitinated proteins, zinc concentration, apoptotic cells, spindle formation, DNA quality, reciprocal translocations, cellular division status, oviductal cell binding, single nucleotide polymorphism (SNP), organelle distribution, data distribution differences, mitochondrial depolarization, and any combination thereof.
71. The method as described in clause 65 or any other clause wherein said regulation of intracellular information comprises information chosen from cAMP, MTOR-pathways, mineral composition, metal, zinc, calcium, cortisol, serotonin, hippocampal glucocorticoid, and any combination thereof.
72. The method as described in clause 65 or any other clause wherein said reduction-oxidation balance comprises information chosen from total antioxidant capacity of cells, total antioxidant capacity of said embryo, total antioxidant capacity of fluids, total antioxidant capacity intracellular, total antioxidant capacity extracellular, presence of oxidants intracellular, presence of oxidants extracellular, membrane reduction oxidative balance, oxidative damage, reactive oxygen species, presence of CD9, oxidative damage, reactive oxygen species, presence of reactive nitrogen species, presence of reactive sulfur species, and any combination thereof.
73. The method as described in clause 65 or any other clause wherein said population of cells comprises information about said embryo chosen from intensity of fluorescence of membrane, intensity of fluorescence of DNA, intensity of fluorescence of acrosomes, intensity of fluorescence of membrane fluidity, delta between fluorescent populations, and any combination thereof.
74. The method as described in clause 65 or any other clause wherein said gamete developmental conditions comprises information chosen from temperature during oogenesis, humidity during oogenesis, temperature during insemination, temperature during culture, temperature of solutions, barometric conditions, and any combination thereof.
75. The method as described in clause 65 or any other clause wherein said personnel skills comprises information chosen from a technician's skills when thawing said embryo, a technician's skills when handling said embryo, a technician's skills when warming said embryo, a technician's skills when implanting said embryo, and any combination thereof.
76. The method as described in clause 61 or any other clause wherein said embryo success rate comprises a parameter chosen from implantation chance, pregnancy retention, parturition rate, total number of animals born alive, and total number of animals born.
77. The method as described in clause 76 or any other clause wherein said embryo success rate comprises a parameter chosen from calving rate, foaling rate, farrowing rate, kidding rate, development of embryo, embryo quality, post-thaw embryo health, embryo transplant success, embryo transfer success, superovulation embryo transfer success, fecundity, fecundability, and any combination thereof.
78. The method as described in clause 61 or 62 or any other clause wherein said step of predicting said embryo success rate of said embryo based on said prediction models completed prediction output comprises a step of generating a numeric indication of said fertility-related parameters.
79. The method as described in clause 78 or any other clause wherein said step of generating said numeric indication of said fertility-related parameters comprises a step of utilizing a r² value from a regression analysis to predict said embryo success rate.
80. The method as described in clause 78 or 79 or any other clause wherein said step generating said numeric indication of said fertility-related parameters comprises a step of evaluating an angle of a line in comparison to a perfect 45-degree angle from a regression analysis computation and utilizing said comparison to predict said embryo success rate.
81. The method as described in clause 80 or any other clause and further comprising the step of combining said r² value and said angle of said line in said comparison to a perfect 45-degree angle to create a combined score and using said combined score in said step of utilizing said comparison to predict said embryo success rate.
82. The method as described in clause 78 or any other clause wherein said step of generating said numeric indication of said fertility-related parameters comprises a step of utilizing variances to predict said fertility-related parameters.
83. The method as described in clause 82 or any other clause wherein said variances are chosen from variances relative to another population, variances relative to populations within a sample, variances relative to other samples taken from a same animal, variances relative to samples taken from related animals, variances within a population, and any combination thereof.
84. The method as described in clause 78 or any other clause wherein said step of generating a numeric indication of said fertility-related parameters comprises using a mathematical property chosen from Rho correlation, delta of means, accuracy, precision, contingency table, and any combination thereof.
85. The method as described in clause 61 or any other clause wherein said animal is chosen from bovine, equine, ovine, porcine, caprine, avian, and human.
86. The method as described in clause 61 or any other clause wherein said prediction models automated computational transformation algorithm comprises a trained, automatically self-improving algorithm based on existing data.
87. The method as described in clause 61 or any other clause and further comprising a step of using one or more different embryo characteristics with said prediction models automated computational algorithm.
88. The method as described in clause 61 or any other clause wherein said embryo is in a blastocyst stage.
89. A method of efficient fertility prediction comprising the steps of:
   evaluating qualities from a male animal and a female animal, wherein said qualities are chosen from gamete type, animal information, gross gamete information, morphological aspects, cellular motion, cellular function, regulation of intracellular information, reduction-oxidation balance, population of cells, gamete developmental conditions, personnel skills, and any combination thereof;
   establishing in a computational device prediction models automated computational transformation algorithm;
   automatically applying said prediction models automated computational transformation algorithm to said qualities to automatically create prediction model transformed data;
   generating prediction models completed prediction output based on said prediction model transformed data of said qualities; and
   predicting fertility-related parameters of said female or male animal based on said prediction models completed prediction output.
90. The method as described in clause 89 or any other clause and further comprising the step of using said fertility-related parameters in making a decision based on said fertility-related parameters.
91. The method as described in clause 91 or any other clause wherein said decision is chosen from a breeding decision, a culling decision, and a type of assisted reproductive technology for said female or male animal.
92. The method as described in clause 89 or any other clause wherein said prediction models automated computational transformation algorithm comprises models chosen from statistical models, mathematical models, machine learning models, regression analyses, and any combination thereof.
93. The method as described in clause 89 or any other clause wherein said gamete type is chosen from fresh, frozen, cooled, flushed embryo, retrieved oocyte, superovulated oocyte, flushed oocyte, fresh sperm, frozen sperm, cooled sperm, vitrified sperm, sperm in gel state, thawed sperm, extended sperm, warmed sperm, freeze dried sperm, dehydrated sperm, rehydrated sperm, in vitro sperm, epidydimal sperm, multiple sperm cells, singular sperm cells, extended semen, and any combination thereof.
94. The method as described in clause 89 or any other clause wherein said animal information comprises information chosen from weight, nutritional status, body condition score, parturition information, breeding schedule, parity, transportation, stress of said animal, synchronization protocol, hemoglobin, fibronectin, inflammation markers, method of collection of oocytes, concentration, hormone levels, donor information, recipient information, temperature, and any combination thereof.
95. The method as described in clause 89 or any other clause wherein said gross gamete information comprises information chosen from seminal plasma information, follicular fluid, electrical conductivity of fluid, storage period, storage temperature, refractometry, thermoresistance, type of solutions, solution characteristics, contaminants, contaminant concentration, organism contaminating, synchronization protocol, follicle health, follicle maturity, oocyte health, and any combination thereof.
96. The method as described in clause 89 or any other clause wherein said morphological aspects comprises information chosen from physical characteristics of cells, percentage abnormal cells, shape descriptions, volume, aspect ratios, ratios of attributes to one another, total mass, mitochondrial distribution, organelle distribution, concentration relative to seminal plasma, percentage of normal cells, length, width, area, thickness, midpiece defects, abnormal heads, distal midpiece reflex, and any combination thereof.
97. The method as described in clause 89 or any other clause wherein said cellular motion comprises information chosen from total motility, progressive motility, velocity descriptors, rate of motility, velocity of motility, percentage of cells in each velocity category, kinematic parameters, mean, median and mode of kinematic parameters, agglutination, and any combination thereof.
98. The method as described in clause 89 or any other clause wherein said cellular function comprises information chosen from acrosome quality, membrane quality, membrane fluidity, mitochondrial quality and depolarization, presence of aggresomes, ubiquitin, ubiquitinated proteins, zinc, zinc concentration, apoptotic cells, spindle formation, DNA quality, reciprocal translocations, cellular division status, oviductal cell binding, single nucleotide polymorphism (SNP), seminal plasma proteins, organelle distribution, data distribution differences, mitochondrial depolarization, and any combination thereof.
99. The method as described in clause 89 or any other clause wherein said regulation of intracellular information comprises information chosen from cAMP, MTOR-pathways, mineral composition, metal, zinc, calcium, cortisol, serotonin, hippocampal glucocorticoid, and any combination thereof.
100. The method as described in clause 89 or any other clause wherein said reduction-oxidation balance comprises information chosen from total antioxidant capacity of cells, total antioxidant capacity of fluids, total antioxidant capacity intracellular, total antioxidant capacity extracellular, presence of oxidants intracellular, presence of oxidants extracellular, membrane reduction oxidative balance, oxidative damage, reactive oxygen species, and any combination thereof.
101. The method as described in clause 89 or any other clause wherein said population of cells comprises information chosen from intensity of fluorescence of membrane, intensity of fluorescence of DNA, intensity of fluorescence of acrosomes, intensity of fluorescence of membrane fluidity, delta between fluorescent populations, median of fluorescence intensity for a specific population, mode of fluorescence intensity for a specific population, and any combination thereof.
102. The method as described in clause 89 or any other clause wherein said developmental conditions comprises information chosen from temperature during spermatogenesis, temperature during spermiogenesis, humidity during spermatogenesis, humidity during spermiogenesis, temperature during oogenesis, humidity during oogenesis, temperature during insemination, temperature during culture, temperature of solutions, barometric conditions, and any combination thereof.
103. The method as described in clause 89 or any other clause wherein said personnel skills comprises information chosen from a technician's skills when thawing cells, a technician's skills when handling cells, a technician's skills when warming cells, a technician's skills when inseminating cells, a technician's skills when implanting cells, and any combination thereof.
104. The method as described in clause 89 or any other clause wherein said fertility-related parameters comprises a parameter chosen from conception rate, parturition rate, total number of animals born alive, and total number of animals born.
105. The method as described in clause 104 or any other clause wherein said fertility-related parameters comprises a parameter chosen from calving rate, foaling rate, farrowing rate, kidding rate, oocyte quality, oocyte fertility potential, oocyte health, post-thaw oocyte health, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, and any combination thereof.
106. The method as described in clause 89 or 91 or any other clause wherein said step of predicting fertility-related parameters of said female or male animal based on said prediction models completed prediction output comprises a step of generating a numeric indication of said fertility-related parameters.
107. The method as described in clause 106 or any other clause wherein said step of generating said numeric indication of said fertility-related parameters comprises a step of utilizing a r² value from a regression analysis to predict said fertility-related parameters.
108. The method as described in clause 106 or 107 or any other clause wherein said step of generating said numeric indication of said fertility-related parameters comprises a step of evaluating an angle of a line in comparison to a perfect 45-degree angle from a regression analysis and utilizing said comparison to predict said fertility-related parameters.
109. The method as described in clause 108 or any other clause and further comprising the step of combining said r² value and said angle of said line in said comparison to a perfect 45-degree angle to create a combined score and using said combined score in said step of utilizing said comparison to predict said fertility-related parameters.
110. The method as described in clause 106 or any other clause wherein said step of generating said numeric indication of said fertility-related parameters comprises a step of utilizing variances to predict said fertility-related parameters.
111. The method as described in clause 110 or any other clause wherein said variances are chosen from variances relative to another population, variances relative to populations within a sample, variances relative to other samples taken from a same animal, variances relative to samples taken from related animals, variances within a population, and any combination thereof.
112. The method as described in clause 106 or any other clause wherein said step of generating a numeric indication of said fertility-related parameters comprises using a mathematical property chosen from Rho correlation, delta of means, accuracy, precision, contingency table, and any combination thereof.
113. The method as described in clause 89 or any other clause wherein said animal is chosen from bovine, equine, ovine, porcine, caprine, avian, and human.
114. The method as described in clause 89 or any other clause wherein said prediction models automated computational transformation algorithm comprises a trained, automatically self-improving algorithm based on existing data.
115. The method as described in clause 89 or any other clause and further comprising a step of using one or more different qualities with said prediction models automated computational transformation algorithm.
116. A method of efficient fertility prediction comprising the steps of:
   evaluating intracellular or extracellular qualities of a reproductive cell;
   establishing in a computational device prediction models automated computational transformation algorithm;
   automatically applying said prediction models automated computational transformation algorithm to said reproductive cell qualities to automatically create prediction model transformed data;
   generating prediction models completed prediction output based on said prediction model transformed data of said reproductive cell qualities; and
   predicting fertility-related parameters of said reproductive cell based on said prediction models completed prediction output.
117. The method as described in clause 116 or any other clause wherein said reproductive cell is chosen from stem cells, pluripotent stem cells, gametes, sperm cells, oocytes, embryos, haploid cells, spermatozoon, ovum, somatic cell nuclear transfer results, small cell nuclear transfer results, and parthenogenic embryos.
118. The method as described in clause 116 or any other clause and further comprising the step of using said fertility-related parameters in making a decision based on said fertility-related parameters.
119. The method as described in clause 118 or any other clause wherein said decision is chosen from a breeding decision, a culling decision, and a type of assisted reproductive technology.
120. The method as described in clause 116 or any other clause wherein said prediction models automated computational transformation algorithm comprises models chosen from statistical models, mathematical models, machine learning models, regression analyses, and any combination thereof.
121. The method as described in clause 116 or any other clause wherein said intracellular or extracellular qualities are chosen from gamete type, animal information, gross gamete information, morphological aspects, cellular function, regulation of intracellular information, reduction-oxidation balance, population of cells, developmental conditions, personnel skills, and any combination thereof.
122. The method as described in clause 121 or any other clause wherein said gamete type is chosen from fresh, frozen, cooled, flushed embryo, retrieved oocyte, superovulated oocyte, flushed oocyte, fresh sperm, frozen sperm, cooled sperm, vitrified sperm, sperm in gel state, thawed sperm, extended sperm, warmed sperm, freeze dried sperm, dehydrated sperm, rehydrated sperm, in vitro sperm, epidydimal sperm, multiple sperm cells, singular sperm cells, and any combination thereof.
123. The method as described in clause 121 or any other clause wherein said animal information comprises information chosen from weight, nutritional status, body condition score, parturition information, breeding schedule, parity, transportation, stress of said animal, synchronization protocol, hemoglobin, fibronectin, inflammation markers, method of collection of oocytes, concentration, hormone levels, donor information, recipient information, temperature, and any combination thereof.
124. The method as described in clause 121 or any other clause wherein said gross gamete information comprises information chosen from seminal plasma information, follicular fluid, electrical conductivity of fluid, storage period, storage temperature, refractometry, thermoresistance, type of solutions, solution characteristics, contaminants, contaminant concentration, organism contaminating, synchronization protocol, follicle health, follicle maturity, oocyte health, and any combination thereof.
125. The method as described in clause 121 or any other clause wherein said morphological aspects comprises information chosen from physical characteristics of cells, percentage abnormal cells, shape descriptions, volume, aspect ratios, ratios of attributes to one another, total mass, mitochondrial distribution, organelle distribution, concentration relative to seminal plasma, percentage of normal cells, length, width, area, thickness, midpiece defects, abnormal heads, distal midpiece reflex, and any combination thereof.
126. The method as described in clause 121 or any other clause wherein said cellular function comprises information chosen from acrosome quality, membrane quality, membrane fluidity, mitochondrial quality and depolarization, presence of aggresomes, ubiquitin, ubiquitinated proteins, zinc, zinc concentration, apoptotic cells, spindle formation, DNA quality, reciprocal translocations, cellular division status, oviductal cell binding, single nucleotide polymorphism (SNP), seminal plasma proteins, organelle distribution, data distribution differences, mitochondrial depolarization, and any combination thereof
127. The method as described in clause 121 or any other clause wherein said regulation of intracellular information comprises information chosen from cAMP, MTOR-pathways, mineral composition, metal, zinc, calcium, cortisol, serotonin, hippocampal glucocorticoid, and any combination thereof.
128. The method as described in clause 121 or any other clause wherein said reduction-oxidation balance comprises information chosen from total antioxidant capacity of cells, total antioxidant capacity of fluids, total antioxidant capacity intracellular, total antioxidant capacity extracellular, presence of oxidants intracellular, presence of oxidants extracellular, membrane reduction oxidative balance, oxidative damage, reactive oxygen species, and any combination thereof.
129. The method as described in clause 121 or any other clause wherein said population of cells comprises information chosen from intensity of fluorescence of membrane, intensity of fluorescence of DNA, intensity of fluorescence of acrosomes, intensity of fluorescence of membrane fluidity, delta between fluorescent populations, median of fluorescence intensity for a specific population, mode of fluorescence intensity for a specific population, and any combination thereof.
130. The method as described in clause 121 or any other clause wherein said developmental conditions comprises information chosen from temperature during spermatogenesis, temperature during spermiogenesis, humidity during spermatogenesis, humidity during spermiogenesis, temperature during oogenesis, humidity during oogenesis, temperature during insemination, temperature during culture, temperature of solutions, barometric conditions, and any combination thereof.
131. The method as described in clause 121 or any other clause wherein said personnel skills comprises information chosen from a technician's skills when thawing cells, a technician's skills when handling cells, a technician's skills when warming cells, a technician's skills when inseminating cells, a technician's skills when implanting cells, and any combination thereof.
132. The method as described in clause 116 or any other clause wherein said fertility-related parameters comprises a parameter chosen from conception rate, parturition rate, total number of animals born alive, and total number of animals born.
133. The method as described in clause 116 or any other clause wherein said fertility-related parameters comprises a parameter chosen from calving rate, foaling rate, farrowing rate, kidding rate, oocyte quality, oocyte fertility potential, oocyte health, post-thaw oocyte health, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, and any combination thereof.
134. The method as described in clause 116 or 120 or any other clause wherein said step of predicting fertility-related parameters of said reproductive cell based on said prediction models completed prediction output comprises a step of generating a numeric indication of said fertility-related parameters.
135. The method as described in clause 134 or any other clause wherein said step of generating said numeric indication of said fertility-related parameters comprises a step of utilizing a r² value from a regression analysis to predict said fertility-related parameters.
136. The method as described in clause 134 or 135 or any other clause wherein said step of generating said numeric indication of said fertility-related parameters comprises a step of evaluating an angle of a line in comparison to a perfect 45-degree angle from a regression analysis and utilizing said comparison to predict said fertility-related parameters.
137. The method as described in clause 136 or any other clause and further comprising the step of combining said r² value and said angle of said line in said comparison to a perfect 45-degree angle to create a combined score and using said combined score in said step of utilizing said comparison to predict said fertility-related parameters.
138. The method as described in clause 134 or any other clause wherein said step of generating said numeric indication of said fertility-related parameters comprises a step of utilizing variances to predict said fertility-related parameters.
139. The method as described in clause 138 or any other clause wherein said variances are chosen from variances relative to another population, variances relative to populations within a sample, variances relative to other samples taken from a same animal, variances relative to samples taken from related animals, variances within a population, and any combination thereof.
140. The method as described in clause 134 or any other clause wherein said step of generating a numeric indication of said fertility-related parameters comprises using a mathematical property chosen from Rho correlation, delta of means, accuracy, precision, contingency table, and any combination thereof.
141. The method as described in clause 116 or any other clause wherein said reproductive cells are taken from an animal chosen from bovine, equine, ovine, porcine, caprine, avian, and human.
142. The method as described in clause 116 or any other clause wherein said prediction models automated computational transformation algorithm comprises a trained, automatically self-improving algorithm based on existing data.
143. The method as described in clause 116 or any other clause and further comprising a step of using one or more different qualities with said prediction models automated computational transformation algorithm.
144. An efficient fertility predictor comprising
   a computational device;
   a computational device prediction models automated computational transformation algorithm on said computational device;
   an input comprising semen qualities from an ejaculate of a male animal; a prediction model data transform of said input with said computational device prediction models automated computational transformation algorithm;
   a prediction models completed prediction output based on said prediction model data transform of said input; and
   predicted fertility-related parameters of said ejaculate of said male animal based on said prediction models completed prediction output.
145. An efficient fertility predictor comprising
   a computational device;
   a computational device prediction models automated computational transformation algorithm on said computational device;
   an input comprising female characteristics of a female animal;
   a prediction model data transform of said input with said computational device prediction models automated computational transformation algorithm;
   a prediction models completed prediction output based on said prediction model data transform of said input; and
   predicted fertility-related parameters of said female animal based on said prediction models completed prediction output.
146. An efficient fertility predictor comprising
   a computational device;
   a computational device prediction models automated computational transformation algorithm on said computational device;
   an input comprising embryo characteristics of an embryo;
   a prediction model data transform of said input with said computational device prediction models automated computational transformation algorithm;
   a prediction models completed prediction output based on said prediction model data transform of said input; and
   predicted an embryo success rate of said embryo based on said prediction models completed prediction output.
147. An efficient fertility predictor comprising
   a computational device;
   a computational device prediction models automated computational transformation algorithm on said computational device;
   an input comprising qualities from a male animal and a female animal, wherein said qualities are chosen from gamete type, animal information, gross gamete information, morphological aspects, cellular motion, cellular function, regulation of intracellular information, reduction-oxidation balance, population of cells, gamete developmental conditions, personnel skills, and any combination thereof;
   a prediction model data transform of said input with said computational device prediction models automated computational transformation algorithm;
   a prediction models completed prediction output based on said prediction model data transform of said input; and
   predicted fertility-related parameters of said female or male animal based on said prediction models completed prediction output.
148. An efficient fertility predictor comprising
   a computational device;
   a computational device prediction models automated computational transformation algorithm on said computational device;
   an input comprising intracellular or extracellular qualities of a reproductive cell;
   a prediction model data transform of said input with said computational device prediction models automated computational transformation algorithm;
   a prediction models completed prediction output based on said prediction model data transform of said input; and
   predicted fertility-related parameters of said reproductive cell based on said prediction models completed prediction output.

As can be easily understood from the foregoing, the basic concepts of the present invention may be embodied in a variety of ways. It involves both predicting techniques as well as devices to accomplish the appropriate prediction. In this application, the predicting techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. As one example, terms of degree, terms of approximation, and/or relative terms may be used. These may include terms such as the words: substantially, about, only, or the like. These words and types of words are to be understood in a dictionary sense as terms that encompass an ample or considerable amount, quantity, size, etc. as well as terms that encompass largely but not wholly that which is specified. Further, for this application if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified. In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person of ordinary skill in this field. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes or the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “transform” should be understood to encompass disclosure of the act of “transforming”—whether explicitly discussed or not—and, conversely, were there effectively disclosure of the act of “transforming”, such a disclosure should be understood to encompass disclosure of a “transform” and even a “means for transforming.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components that are configured to, or configured and arranged to, achieve a particular result, use, purpose, situation, function, or operation, or as components that are capable of achieving a particular result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The Provisional Patent Application or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

### REFERENCES TO BE INCORPORATED BY REFERECE

### U.S. PATENTS

| | | Name of Patentee or Applicant |
|---|---|---|
| Pat. No. | Issue Date | of cited Document |
| 9,458,506 | 2016 Oct. 4 | Chavez et al. |
| 10,162,800 | 2018 Dec. 25 | Elashoff et al. |

### NONPATENT LITERATURE

U.S. Provisional Pat. Application No. 62/908,743, filed Oct. 1, 2020. First
Named Inventor: Herickhoff.
U.S. Provisional Pat. Application No. 63/049,608, filed Jul. 8, 2020. First
Named Inventor: Herickhoff.
Long, et al. Estimating Genetic Merit,
https://porkgateway.org/resource/estimating-gentic-merit/. Published
Jun. 3, 2006. 3 pages.
Swalin, Choosing the Right Metric for Evaluating Machine Learning
Models - Part 1. https://medium.com/usf-msds/choosing-the-right-metric-
for-machine-learning-models-part-1-a99d7d7414e4. Apr. 7, 2018. 7 pages.

Thus, the applicant(s) should be understood to have support to claim and make a statement of invention to at least: i) each of the prediction devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, it should be understood that in characterizing these and all other aspects of the invention—whether characterized as a device, a capability, an element, or otherwise, because all of these can be implemented via software, hardware, or even firmware structures as set up for a general purpose computer, a programmed chip or chipset, an ASIC, application specific controller, subroutine, or other known programmable or circuit specific structure—it should be understood that all such aspects are at least defined by structures including, as person of ordinary skill in the art would well recognize: hardware circuitry, firmware, programmed application specific components, and even a general purpose computer programmed to accomplish the identified aspect. For such items implemented by programmable features, the applicant(s) should be understood to have support to claim and make a statement of invention to at least: xv) processes performed with the aid of or on a computer, machine, or computing machine as described throughout the above discussion, xvi) a programmable apparatus as described throughout the above discussion, xvii) a computer readable memory encoded with data to direct a computer comprising means or elements which function as described throughout the above discussion, xviii) a computer, machine, or computing machine configured as herein disclosed and described, xix) individual or combined subroutines and programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) a processor executing instructions that act to achieve the steps and activities detailed, xxvi) circuitry configurations (including configurations of transistors, gates, or the like) that act to sequence and/or cause actions as detailed, xxvii) computer readable medium(s) storing instructions to execute the steps and cause activities detailed, xxviii) the related methods disclosed and described, xxix) similar, equivalent, and even implicit variations of each of these systems and methods, xxx) those alternative designs which accomplish each of the functions shown as are disclosed and described, xxxi) those alternative designs and methods which accomplish each of the functions shown as are implicit to accomplish that which is disclosed and described, xxxii) each feature, component, and step shown as separate and independent inventions, and xxxiii) the various combinations of each of the above and of any aspect, all without limiting other aspects in addition.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, or the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, or the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method of efficient fertility prediction comprising the steps of:
evaluating semen qualities from an ejaculate of a male animal;
predicting male fertility-related parameters of said ejaculate of said male animal; and
using said male fertility-related parameters in making a decision based on said male fertility-related parameters;
wherein said semen qualities are chosen from cellular motion, cellular function, regulation of intracellular information, and reduction-oxidation balance;
wherein said decision is chosen from a breeding decision, a culling decision, and a type of assisted reproductive technology.

2. A method of efficient fertility prediction comprising the steps of:
evaluating semen qualities from an ejaculate of a male animal;
establishing in a computational device prediction models automated computational transformation algorithm;
automatically applying said prediction models automated computational transformation algorithm to said semen qualities to automatically create prediction model transformed data of said semen qualities;
generating prediction models completed prediction output based on said prediction model transformed data of said semen qualities;
predicting male fertility-related parameters of said ejaculate of said male animal based on said prediction models completed prediction output; and
using said male fertility-related parameters in making a decision based on said male fertility-related parameters;
wherein said semen qualities are chosen from cellular motion, cellular function, regulation of intracellular information, and reduction-oxidation balance;
wherein said decision is chosen from a breeding decision, a culling decision, and a type of assisted reproductive technology.

3. The method as described in claim 2 wherein said prediction models automated computational transformation algorithm comprises models chosen from statistical models, mathematical models, machine learning models, regression analyses, and any combination thereof.

4-5. (canceled)

6. The method as described in claim 1 wherein said semen qualities are chosen from semen state, animal information of said male animal, gross sperm information, morphological aspects, population of cells, sperm developmental conditions, personnel skills, and any combination thereof.

7-10. (canceled)

11. The method as described in claim 1 wherein said cellular motion comprises information chosen from total motility, progressive motility, velocity descriptors, rate of motility, velocity of motility, percentage of cells in each velocity category, kinematic parameters, mean, median and mode of kinematic parameters, agglutination, and any combination thereof.

12. The method as described in claim 1 wherein said cellular function comprises information chosen from acrosome quality, membrane quality, membrane fluidity, mitochondrial quality and depolarization, presence of aggresomes, ubiquitin, ubiquitinated proteins, zinc, zinc concentration, apoptotic cells, DNA quality, reciprocal translocations, single nucleotide polymorphism (SNP), seminal plasma proteins, data distribution differences, mitochondrial depolarization, and any combination thereof.

13. The method as described in claim 1 wherein said regulation of intracellular information comprises information chosen from cAMP, MTOR-pathways, mineral composition, metal, zinc, calcium, cortisol, serotonin, hippocampal glucocorticoid, and any combination thereof.

14. The method as described in claim 1 wherein said reduction-oxidation balance comprises information chosen from total antioxidant capacity of cells, total antioxidant capacity of extender, total antioxidant capacity of seminal plasma, total antioxidant capacity intracellular, total antioxidant capacity extracellular, superoxide dismutase concentration, endogenous and exogenous antioxidants, presence of oxidants intracellular, presence of oxidants extracellular, presence of antioxidants intracellular, presence of antioxidants extracellular, membrane reduction-oxidation balance, oxidative damage, reactive oxygen species, reactive sulfur species, reactive nitrogen species, and any combination thereof.

15-17. (canceled)

18. The method as described in claim 1 wherein said fertility-related parameters comprises a parameter chosen from conception rate, parturition rate, total number of animals born alive, and total number of animals born.

19. The method as described in claim 18 wherein said fertility-related parameters comprises a parameter chosen from calving rate, foaling rate, farrowing rate, kidding rate, development of embryo, embryo quality, post-thaw embryo health, embryo transplant success, embryo transfer success, superovulation fertilization success, superovulation embryo transfer success, intracytoplasmic sperm injection success, fecundity, fecundability, infertility, sub-fertility, delayed fertility, and any combination thereof.

20. The method as described in claim 3 wherein said step of predicting said fertility-related parameters of said ejaculate of said male animal based on said prediction models completed prediction output comprises a step of generating a numeric indication of said fertility-related parameters.

21. The method as described in claim 20 wherein said step of generating said numeric indication of said fertility-related parameters comprises a step of utilizing a r² value from a regression analysis to predict said fertility-related parameters.

22. The method as described in claim 21 wherein said step of generating said numeric indication of said fertility-related parameters comprises a step of evaluating an angle of a line in comparison to a perfect 45-degree angle from a regression analysis and utilizing said comparison to predict said fertility-related parameters.

23. The method as described in claim 22 and further comprising the step of combining said r² value and said angle of said line in said comparison to a perfect 45-degree angle to create a combined score and using said combined score in said step of utilizing said comparison to predict said fertility-related parameters.

24. The method as described in claim 20 wherein said step of generating said numeric indication of said fertility-related parameters comprises a step of utilizing variances to predict said fertility-related parameters.

25. The method as described in claim 24 wherein said variances are chosen from variances relative to another population, variances relative to populations within a sample, variances relative to other samples taken from a same animal, variances relative to samples taken from related animals, variances within a population, and any combination thereof.

26. The method as described in claim 20 wherein said step of generating a numeric indication of said fertility-related parameters comprises using a mathematical property chosen from Rho correlation, delta of means, accuracy, precision, contingency table, and any combination thereof.

27. The method as described in claim 2 wherein said animal is chosen from bovine, equine, ovine, porcine, caprine, avian, and human.

28-29. (canceled)

30. The method as described in claim 2 and further comprising the steps of:
evaluating female characteristics from a female animal;
automatically applying said prediction models automated computational transformation algorithm to said female characteristics to automatically create said prediction model transformed data of said semen qualities and said female characteristics; and
generating said prediction models completed prediction output based on said prediction model transformed data of said semen qualities and said female characteristics.

31-148. (canceled)

149. The method as described in claim 1 wherein said semen qualities exclude genetic biomarker information.

150. (canceled)
